(19) United States
(12) Patent Application Publication
(10) Pub. No.: US 2026/0117278 A1
(43) Pub. Date: Apr. 30, 2026

(54) METHOD, KITS AND SYSTEM FOR DUAL LABELING OF NUCLEIC ACIDS

(71) Applicant: Cheng-Yao CHEN, Hsinchu (TW)

(72) Inventor: Cheng-Yao CHEN, Hsinchu (TW)

(21) Appl. No.: 18/900,997

(22) Filed: Sep. 30, 2024

Related U.S. Application Data
(63) Continuation of application No. PCT/US2023/067563, filed on May 26, 2023.
(60) Provisional application No. 63/346,416, filed on May 27, 2022.
(60) Provisional application No. 63/406,941, filed on Sep. 15, 2022.

(51) Int. Cl. C12Q 1/6806; C12Q 1/44; C12Q 1/48

(52) U.S. Cl. CPC C12Q 1/6806; C12Q 1/44; C12Q 1/48; C12Y 207/07007; C12Y 301/15; C12Y 301/16; C12Y 302/02

(57) ABSTRACT

Provided are methods for introducing a modification to an end of a nucleic acid, thereby labeling the nucleic acid with desired moiety, including to a 3′-end, a 5′-end or both 3′-end and 5′-end of a nucleic acid. Also provided are kits for introducing such modification to ends of a nucleic acid.

### CROSS REFERENCE TO RELATED APPLICATIONS

This application is a continuation application of PCT Application No. PCT/US2023/067563, filed on May 26, 2023, which claims the benefit of U.S. Provisional Application No. 63/346,416, filed on May 27, 2022, and claims the benefit of U.S. Provisional Application No. 63/406,941, filed on Sep. 15, 2022. The contents of these applications are incorporated herein by reference.

### SEQUENCE LISTING

This application contains a Sequence Listing XML, which has been submitted electronically and is hereby incorporated by reference in its entirety. The Sequence Listing XML, created on May 26, 2023, is named “YDBL-0005PCTUS-Sequence Listing-2023-0526.xml” and is 6.2 kb in size.

### BACKGROUND OF THE INVENTION

### 1. Field of the Invention

The present disclosure relates to modifications to both ends of a nucleic acid, more particularly to the modifications at both 3′-end and 5′-end of a nucleic acid.

### 2. Description of the Prior Art

Nucleic acids labeling is routinely implemented in biomedical and biological applications, including identification and purification of unknown or target gene fragments, localization of target gene sequences, pinpointing nucleic acids-protein interaction, and visualization of cell and tissue dynamics. Generally, methods for labeling of nucleic acids can be categorized into chemical or enzymatic approaches. The chemical labeling methods involve the modification of the 5′-phosphate group, 3′-hydroxyl group, nucleobase, or sugar moiety of the target nucleic acids chemical-reactive using compounds, such as N-(3-dimethylaminopropyl)-N′-ethylcarbodiimide (EDC), imidazole, hydrazine, sodium periodate, and sodium cyanoborohydride, to modify the structure of nucleic acids and subsequently attach a chemical or functional moiety to the desired nucleic acids.

On the other hand, the enzymatic nucleic acid labeling methods utilize enzymes, such as alkaline phosphatases, nucleic acid kinases, or DNA/RNA polymerases, to substitute for, add, or incorporate a chemical or functional moiety, such as a radioisotope, a biotin group, or a fluorescent-labeled nucleotide to the target nucleic acids, thereby attaching a desired label to the nucleic acids.

Although a plethora of nucleic acid labeling techniques are available to generate nucleic acid probes such as those labeled with fluorophores, enzymes, and radioactive phosphates, or nucleotides modified with digoxigenin or biotin, these methods normally have lengthy and complicated procedures and make use of many types of enzymes, reactive chemicals, or radioactive isotopes, which often require specialized enzymes, chemicals, or personal training on handling of toxic or radioactive materials and wastes, making the nucleic acid labeling a tedious and inefficient process.

Furthermore, the labeling efficiency of existing nucleic acid labeling methods varies depending on the length and type of nucleic acids, the position of targeted site to be labeled (e.g., the internal or terminal nucleotide(s) of a nucleic acid sequence), as well as the chemical or functional moieties to be labeled. Therefore, there is still an unmet need for simple, efficient, and eco-friendly nucleic acid labeling methods.

### SUMMARY OF THE INVENTION

The present disclosure provides a method for efficiently modifying or labeling the 3′-end of nucleic acids or polynucleotides. The method deploys a pair of functional moieties or chemical molecules that can stably or covalently couple with each other, thereby introducing a specific modification or label to the 3′-end of nucleic acids or polynucleotides. For example, one component of the paired functional moieties or chemical molecules is labeled on a nucleobase or the 3′-hyodroxyl (3′-OH) group of a nucleotide, which is incorporated by a polymerase to the 3′-end of the target nucleic acid or polynucleotide. The resulting target nucleic acids or polynucleotides contain the first functional moiety or chemical molecule at the 3′-end, which can readily react with the other component of the paired moieties or molecules carrying a desired modification or label. Consequently, the reaction between the paired functional moieties forms a stable or covalent linkage, such that the target nucleic acids or polynucleotides can be modified or labeled with the desired molecule, especially at the 3′-end of the target nucleic acids or polynucleotides.

In at least one embodiment, the method provided by the present disclosure comprises modifying a natural or synthetic deoxyribonucleic acid (DNA) at the 3′-end. FIG. 1 shows a schematic diagram depicting an exemplary method of introducing a 3′-modification to a target nucleic acid. In some embodiments, the method provided by the present disclosure comprises providing a polynucleotide including a 3′-end nucleotide (e.g., “Nucleotide” as illustrated in FIG. 1) having a reactive moiety (e.g., “M1” as illustrated in FIG. 1); and exposing the polynucleotide to a desired molecule (e.g., “Label” as illustrated in FIG. 1) having a corresponding functional moiety (e.g., “M2” as illustrated in FIG. 1) capable of reacting with the reactive moiety to form a linkage, thereby coupling the desired molecule to the 3′-end of the polynucleotide. In some embodiments, the desired molecule has a label moiety (e.g., “Label” as illustrated in FIG. 1) being introduced into the polynucleotide to form a labeled polynucleotide.

In at least one embodiment, the method provided by the present disclosure further comprises preparing the polynucleotide by a template-independent enzymatic nucleic acid synthesis. In some embodiments, the template-independent enzymatic nucleic acid synthesis comprises employing a DNA polymerase, an RNA polymerase, or a functionally equivalent enzyme thereof. In some embodiments, the DNA polymerase of the template-independent enzymatic nucleic acid synthesis is an A family DNA polymerase, a B family DNA polymerase, or an X family DNA polymerase. In at least one embodiment, the B family DNA polymerase is a Thermococcaceae DNA polymerase. In at least one embodiment, the B family DNA polymerase is a Thermococcus or a Pyrococcus DNA polymerase. In at least an embodiment, the B family DNA polymerase is selected from the group consisting of a B family DNA polymerase of Thermococcus kodakarensis (Kod1), a B family DNA polymerase of Pyrococcus furiosus (Pfu), a B family DNA polymerase of Thermococcus litoralis (Vent), a B family DNA polymerase of Thermococcus sp. 9°N (9°N), and a B family DNA polymerase of Thermococcus gorgonarius (Tgo).

In at least one embodiment of the present disclosure, the template independent enzymatic nucleic acid synthesis is performed at a reaction temperature of from 10° C. to 100° C., such as 10° C. to 90° C., 20° C. to 90° C., 30° C. to 90° C., 20° C. to 80° C., 30° C. to 80° C., 40° C. to 80° C., 30° C. to 70° C., 40° C. to 70° C., or 50° C. to 70° C.

In some embodiments, the method provided by the present disclosure further comprises preparing the polynucleotide in a solution phase. In other embodiments, the method provided by the present disclosure comprises preparing the polynucleotide in a solid phase, e.g., providing an initiator attached to a solid support. In some embodiments, the solid support is selected from the group consisting of a particle, a polymer, a bead, a resin, a slide, a chip, an array surface, a membrane, a flow cell, a well, a matrix, a chamber, a microfluidic chamber, a channel, a microfluidic channel, and a gel.

In at least one embodiment, the method provided by the present disclosure further comprises providing an endonuclease to enzymatically release the polynucleotide from the initiator. In some embodiments, the endonuclease recognizes the 3′-penultimate nucleotide of the initiator and cleaves a linkage bond between the 3′-end nucleotide of the initiator and the polynucleotide, between the 3′-penultimate nucleotide and a 3′-antepenultimate nucleotide of the initiator, between a 3′-antepenultimate nucleotide and a 3′-preantepenultimate nucleotide of the initiator, or between a 3′-preantepenultimate nucleotide and a 3′-propreantepenultimate nucleotide of the initiator.

In at least one embodiment of the present disclosure, the endonuclease is derived from Thermococcus barophilus (Tba), Pyrococcus furiosus (Pfu), Methanosarcina acetivorans (Mac), Pyrococcus abyssi (Pab), Thermococcus kodakarensis (Tko), Thermococcus gammatolerans (Tga), or Bacillus subtilis (Bsu).

In at least one embodiment of the present disclosure, the 3′-end nucleotide is a natural nucleotide, a nucleotide analogue, or an abasic (apurinic/apyrimidinic) nucleotide. In some embodiments, the 3′-end nucleotide is a ribonucleotide, a deoxyribonucleotide, or a xeno-nucleotide. In some embodiments, the reactive moiety is linked to the 2′-carbon or 3′-carbon of the nucleosugar, or the nucleobase of the 3′-end nucleotide.

In at least one embodiment of the present disclosure, the corresponding functional moiety reacts with the reactive moiety via a bioorthogonal reaction. In some embodiments, the bioorthogonal reaction is click conjugation, oxime/hydrazine formation, Staudinger ligation, tetrazine ligation, or quadricyclane ligation. In some embodiments, the click conjugation is selected from the group consisting of copper-catalyzed azide-alkyne cycloaddition (CuAAC), strain-promoted azide-alkyne cycloaddition (SPAAC), isocyanide-based click reaction, and inverse electron demand Diels-Alder reaction (IEDDA).

In at least one embodiment of the present disclosure, the reactive moiety is selected from the group consisting of an azido group, an alkynyl group, a triarylphosphinyl group, a cyclooctynyl group, a thiol group, an alkenyl group, a nitrone group, an aldehydyl group, a ketonyl group, a dienyl group, and a dienophilyl group.

In at least one embodiment of the present disclosure, the corresponding functional moiety is a functional group selected from the group consisting of an azido group, an alkynyl group, a triarylphosphinyl group, a cyclooctynyl group, a thiol group, an alkenyl group, a nitrone group, an aldehydyl group, a ketonyl group, a dienyl group, and a dienophilyl group.

In some embodiments, the bioorthogonal reaction is performed at a reaction temperature of from 10° C. to 100° C., such as 10° C. to 90° C., 10° C. to 80° C., 10° C. to 70° C., 10° C. to 60° C., 20° C. to 80° C., 20° C. to 70° C., 20° C. to 60° C., 20° C. to 50° C., 30° C. to 70° C., 30° C. to 60° C., 30° C. to 50° C., or 30° C. to 40° C. In some embodiments, the bioorthogonal reaction is performed for, e.g., 1 min, 5 min, 10 min, 20 min, 30 min, 40 min, 50 min, 1 hr, 2 hr, 5 hr, 10 hr, 12 hr, 16 hr, 24 hr, 36 hr, 48 hr or more.

In at least one embodiment, the desired molecule is molecularly recognizable through detection of visible light, fluorescence, photoluminescence, electrochemiluminescence, laser, irradiation, fluorescence resonance energy transfer, fluorogenic conformational change, or fluorescence quenching. In some embodiments, the desired molecule is a chemical compound, a fluorescent tag, a dye, a marker, a reporter, a quencher, an amine, an antigen, a ligand, a protein, an antibody, an antibody fragment, a peptide, a peptide analog, or a quantum dot.

In at least one embodiment, the method provided by the present disclosure further comprises a clean-up or enrichment step to remove unlabeled nucleic acids or polynucleotides, e.g., providing a protein possessing a 3′ to 5′ exonuclease activity to digest the nucleic acids or polynucleotides with an unsuccessful 3′-end nucleotide synthesis by a polymerase or an incomplete coupling reaction with a second reactive moiety.

In at least one embodiment, the present disclosure also provides a kit for modifying a polynucleotide at its 3′-end, which comprises a nucleotide having a reactive moiety, a polymerase to incorporate the nucleotide having the reactive moiety to the 3′-end of the polynucleotide, and a desired molecule having a corresponding functional moiety capable of reacting with the reactive moiety. In at least one embodiment, the kit for modifying a polynucleotide at its 3′-end comprises a nucleotide having a reactive moiety, a polymerase, a desired labeling molecule, and a 3′ to 5′ exonuclease, wherein the polynucleotide is coupled with the desired labeling molecule at the 3′-end to form a labeled polynucleotide, which can be further enriched by the clean-up exonuclease described herein.

The present disclosure also provides a method for 5′-end labeling of a nucleic acid, the method comprising: providing a target nucleic acid to be labeled; providing a 5′-end glycosylase to react with the target nucleic acid to create an intermediate nucleic acid having an abasic site at a 5′-end of the target nucleic acid; and providing an aldehyde-reactive compound carrying a detectable label for coupling with the intermediate nucleic acid at the abasic site to form a labeled nucleic acid with the detectable label attached at the 5′-end.

In at least an embodiment of the present disclosure, the nucleic acid is single-stranded or comprises at least a duplex region formed by two complimentary strands of nucleic acids. In an embodiment, the nucleic acid is a DNA fragment or an RNA fragment. In another embodiment, the nucleic acid is synthesized de novo or derived from a biological organism. In some embodiments, the nucleic acid is immobilized on a solid-state surface or a polymer surface.

In at least an embodiment of the present disclosure, the aldehyde-reactive compound is a compound having at least one primary amine, a hydrazide, an acylhydrazide, a compound having an aminooxy (ONH₂) group, a compound having a naphthalene-containing aminooxy group, and/or a compound having a guanidine-containing aminooxy group. In some embodiments, the aldehyde-reactive compound is a hydroxylamine biotin, an aminooxy-poly(ethylene glycol)-azide, an propargyl, an aminooxy-poly(ethylene glycol)-DBCO, an aminooxy-poly(ethylene glycol)-bicyclononyne (BCN), a fluorescent dye-hydroxylamine such as Alexa Fluor 488 hydroxylamine, an aldehyde-reactive probe (ARP), an aminooxy-fluorescent dye such as an aminooxy-5(6)-FAM, an aminooxy-5(6)-ROX and an aminooxy-5(6)-TAMRA, a cyanine 555 aminooxy, a cyanine 647 aminooxy, an aminooxy-biotin, a naphthalene-containing aminooxy-fluorescent dye, a guanidine-containing aminooxy-fluorescent dye, a naphthalene- and/or guanidine-containing aminooxy-FAM, a Cy5-PEG-aminooxy, or a fluorescent dye hydrazide such as a cyanine dye hydrazide or a CF dye hydrazide.

In at least an embodiment of the present disclosure, the nucleic acid comprises a 5′-end nucleobase selected from the group consisting of hypoxanthine, cytosine, 3-alkyladenine, 8-oxoguanine (8-oxoG), uracil, 5-hydroxyuracil, 5-hydroxymethyluracil, 5-formyluracil, 5-fluorouracil, dihydroxyuracil, 5-formylcytosine, 5-carboxylcytosine, 3-methyladenine (3-meA), 3-methylguanine, 7-methyladenine, 7-methylguanine, N6-methyladenine, 8-oxo-7,8-dihydroguanine, 5-hydroxylcytosine, ethenocytosine, ethenoadenine, thymine glycol, cytosine glycol, 2,6-diamino-4-hydroxy-5-N-methylformamidopyrimidine, a formamidopyrimidine derivative of adenine, and a formamidopyrimidine derivative of guanine.

In some embodiments, the 5′-end glycosylase of the present disclosure may be a mono-functional DNA glycosylase. In at least an embodiment of the present disclosure, the mono-functional DNA glycosylase may be selected from the group consisting of uracil-DNA glycosylase (UDG or UNG), alkyladenine DNA glycosylase (AAG; also referred to as methylpurine DNA glycosylase (MPG)), single-strand-selective monofunctional uracil-DNA glycosylase 1 (SMUG1), methyl-binding domain glycosylase 4 (MBD4), thymine DNA glycosylase (TDG), MutY homolog DNA glycosylase (MYH), alkylpurine glycosylase C (AlkC), alkylpurine glycosylase D (AlkD), 8-oxo-guanine glycosylase 1 (OGG1) without an abasic site lyase activity, endonuclease III-like glycosylase 1 (NTHL1) without the abasic site lyase activity, endonuclease VIII-like glycosylase 1 (NEIL1) without the abasic site lyase activity, endonuclease VIII-like glycosylase 2 (NEIL2) without the abasic site lyase activity, endonuclease VIII-like glycosylase 3 (NEIL3) without the abasic site lyase activity, enzymatically active fragments thereof and any combination thereof.

In at least an embodiment of the present disclosure, the uracil-DNA glycosylase is derived from the family of Micrococcaceae, Staphylococcaceae, or Caryophanaceae, which includes a genus of Micrococcus, Stomatococcus, Staphylococci, or Planococcus. In some embodiments, the uracil-DNA glycosylase is derived from Micrococcus luteus.

In at least an embodiment of the present disclosure, the detectable label is selected from the group consisting of an azide, an alkyne, a bicyclononyne (BCN), a dibenzocyclooctyne (DBCO), a maleimide, a peptide, a protein, an antibody, a dendrimer, a biotin, a radioisotope, a photochromic dye, a fluorescent dye, a luminescent dye, and any combination thereof.

In some embodiments, the method of the present disclosure further comprises providing a 5′ to 3′ exonuclease to remove unlabeled nucleic acids. In at least an embodiment, the 5′ to 3′ exonuclease is selected from the group consisting of T5 exonuclease (T5 exo), T7 exonuclease (T7 exo), viral alkaline exonuclease, bacterial alkaline exonuclease, phage lambda exonuclease, 5′-exonuclease of DNA polymerase I (ExoVI) from, e.g., Streptococcus pneumoniae or Helicobacter pylori, Escherichia coli exonuclease VIII (Exo VIII), RecJ from, e.g., Escherichia coli or Deinococcus radiodurans, RecJf derived from RecJ fusion to the maltose-binding protein, Thermus thermophilus (Tth) RecJ, Mycoplasma pneumonia (Mpn) NrnA, human exonuclease 5 (hEXO5), human exonuclease 1 (hEXO1), SNM1 from Saccharomyces cerevisiae, human or bovine SNM1A, human SNM1B/Apollo, bovine SNM1B, SXT-Exo from, e.g., Vibrio cholerae, phospholipase D3 (PLD3), phospholipase D4 (PLD4), Sso1391-Csa1 from, e.g., Sulfolobus solfataricus, Sto0027-Csa1 from, e.g., Sulfolobus tokadaii, Ttx1248-Csa1 from, e.g., Thermoproteus tenax, Sso1451-Csa1 from, e.g., Sulfolobus solfataricus, Sto2633-Csa1 from, e.g., Sulfolobus tokadaii, Pfu1793-Cas4 from, e.g., Pyrococcus furiosus, Sto2501 from, e.g., Sulfolobus tokadaii, Sso0001 from, e.g., Sulfolobus solfataricus, Sto2331-Cas4 from, e.g., Sulfolobus tokadaii, Ttx1245-Cas4 from, e.g., Thermoproteus tenax, Sso1449-Cas4 from, e.g., Sulfolobus solfataricus, Sto2635-Cas4 from, e.g., Sulfolobus tokadaii, Sso1392-Cas4 from, e.g., Sulfolobus solfataricus, Sulfolobus islandicus rod-shaped virus 2 (SIRV2) gp19, bacterial AddB, and any combination thereof.

In some embodiments, the method of the present disclosure further comprises a nucleic acid synthesis process to obtain a unique 5′-end nucleobase. In some embodiments, the nucleic acid having a unique 5′-end nucleobase is synthesized by a process well known in the art, including the phosphoramidite-based nucleic acid synthesis process, and the template-dependent and template-independent enzymatic nucleic acid synthesis processes.

In some embodiments, the method of the present disclosure further comprises isolating a nucleic acid fragment from a sample. For example, the nucleic acid may be isolated from a sample of intact or disrupted viruses or cells, such as bacterial, archaeal, and eukaryotic cells, e.g., human cells. Suitable samples include isolated cell and tissue samples, such as biopsies, including the solid tissue or tumor biopsies. In some embodiments, the sample may be obtained from a formalin-fixed paraffin embedded (FFPE) tissue sample or other stored samples of cellular materials.

The present disclosure also provides a kit for 5′-end labeling of a nucleic acid, and the kit comprises a 5′-end glycosylase and an aldehyde-reactive compound.

In some embodiments, the 5′-end glycosylase in the kit of the present disclosure is selected from the group consisting of uracil-DNA glycosylase (UDG or UNG), alkyladenine DNA glycosylase (AAG; also referred to as methylpurine DNA glycosylase (MPG)), single-strand-selective monofunctional uracil-DNA glycosylase 1 (SMUG1), methyl-binding domain glycosylase 4 (MBD4), thymine DNA glycosylase (TDG), MutY homolog DNA glycosylase (MYH), alkylpurine glycosylase C (AlkC), alkylpurine glycosylase D (AlkD), 8-oxo-guanine glycosylase 1 (OGG1) without an abasic site lyase activity, endonuclease III-like glycosylase 1 (NTHL1) without the abasic site lyase activity, endonuclease VIII-like glycosylase 1 (NEIL1) without the abasic site lyase activity, endonuclease VIII-like glycosylase 2 (NEIL2) without the abasic site lyase activity, endonuclease VIII-like glycosylase 3 (NEIL3) without the abasic site lyase activity, enzymatically active fragments thereof, and any combination thereof.

In at least an embodiment, the uracil-DNA glycosylase in the kit of the present disclosure is derived from the family of Micrococcaceae, Staphylococcaceae, or Caryophanaceae, which includes a genus of Micrococcus, Stomatococcus, Staphylococci, or Planococcus. In some embodiments, the uracil-DNA glycosylase is derived from Micrococcus luteus.

In at least an embodiment, the aldehyde-reactive compound in the kit of the present disclosure is a compound having at least one primary amine, a hydrazide, an acylhydrazide, a compound having an aminooxy (—ONH₂) group, and a compound having a naphthalene- and/or guanidine-containing aminooxy group. In some embodiments, the aldehyde-reactive compound is a hydroxylamine biotin, a fluorescent dye-hydroxylamine such as Alexa Fluor 488 hydroxylamine, an aldehyde-reactive probe (ARP), an aminooxy-fluorescent dye such as an aminooxy-5(6)-FAM, an aminooxy-5(6)-ROX and an aminooxy-5(6)-TAMRA, a cyanine 555 aminooxy, a cyanine 647 aminooxy, an aminooxy-biotin, a naphthalene-containing aminooxy-fluorescent dye, a guanidine-containing aminooxy-fluorescent dye, a naphthalene- and/or guanidine-containing aminooxy-FAM, a Cy5-PEG-aminooxy, or a fluorescent dye hydrazide such as a cyanine dye hydrazide or a fluorescent CF dye hydrazide.

In at least an embodiment, the kit of the present disclosure further comprises a 5′ to 3′ exonuclease for removing an unlabeled nucleic acid. In some embodiments, the 5′ to 3′ exonuclease is selected from the group consisting of T5 exonuclease, T7 exonuclease, phage lambda exonuclease, 5′-exonuclease of DNA polymerase I (ExoVI), exonuclease VIII (Exo VIII), RecJ, RecJf, Tth RecJ, Mpn NrnA, human EXO5 (hEXO5), human exonuclease 1 (hEXO1), SNM1, SNM1A, human SNM1B/Apollo, bovine SNM1B, SXT-Exo, phospholipase D3 (PLD3), phospholipase D4 (PLD4), Sso1391-Csa1, Sto0027-Csa1, Ttx1248-Csa1, Sso1451-Csa1, Sto2633-Csa1, Pfu1793-Cas4, Sto2501, Sso0001, Sto2331-Cas4, Ttx1245-Cas4, Sso1449-Cas4, Sto2635-Cas4, Sso1392-Cas4, SIRV2 gp19, bacterial AddB, and any combination thereof.

The present disclosure further provides a system for 5′-end labeling of a nucleic acid, the system comprising a reaction reservoir, chamber or vessel, a liquid handling/transferring device, a temperature control unit, and a time control unit, wherein the liquid handling/transferring device is configured to transfer a 5′-end glycosylase and an aldehyde-reactive compound to a nucleic acid in the reaction reservoir, chamber or vessel for a period of time at a defined temperature, which is controlled by the temperature control unit.

The present disclosure further provides a kit for 5′-end labeling of a nucleic acid, the kit comprising a 5′-end glycosylase and an aldehyde-reactive compound. In some embodiments, the kit further comprises a 5′ to 3′ exonuclease for removing an unlabeled nucleic acid, such as T5 exonuclease, T7 exonuclease, bacterial alkaline exonuclease, viral alkaline exonuclease, phage lambda exonuclease, 5′-exonuclease of DNA polymerase I (ExoVI), exonuclease VIII (Exo VIII), RecJ, RecJf, Tth RecJ, Mpn NrnA, human exonuclease 5 (hEXO5), human exonuclease 1 (hEXO1), SNM1, SNM1A, human SNM1B/Apollo, bovine SNM1B, SXT-Exo, phospholipase D3 (PLD3), phospholipase D4 (PLD4), Sso1391-Csa1, Sto0027-Csa1, Ttx1248-Csa1, Sso1451-Csa1, Sto2633-Csa1, Pfu1793-Cas4, Sto2501, Sso0001, Sto2331-Cas4, Ttx1245-Cas4, Sso1449-Cas4, Sto2635-Cas4, Sso1392-Cas4, SIRV2 gp19, bacterial AddB, and any combination thereof.

In at least an embodiment, the 5′-end glycosylase is selected from the group consisting of uracil-DNA glycosylase (UDG or UNG), alkyladenine DNA glycosylase (AAG), single-strand-selective monofunctional uracil-DNA glycosylase 1 (SMUG1), methyl-binding domain glycosylase 4 (MBD4), thymine DNA glycosylase (TDG), MutY homolog DNA glycosylase (MYH), alkylpurine glycosylase C (AlkC), alkylpurine glycosylase D (AlkD), 8-oxo-guanine glycosylase 1 (OGG1) without an abasic site lyase activity, endonuclease III-like glycosylase 1 (NTHL1) without the abasic site lyase activity, endonuclease VIII-like glycosylase 1 (NEIL1) without the abasic site lyase activity, endonuclease VIII-like glycosylase 2 (NEIL2) without the abasic site lyase activity, endonuclease VIII-like glycosylase 3 (NEIL3) without the abasic site lyase activity, enzymatically active fragments thereof, and any combination thereof. In at least an embodiment, the uracil-DNA glycosylase is derived from a family of Micrococcaceae, Staphylococcaceae, or Caryophanaceae.

In at least an embodiment, the aldehyde-reactive compound is a hydroxylamine biotin, a fluorescent dye-hydroxylamine, an aldehyde-reactive probe (ARP), an aminooxy-fluorescent dye, an aminooxy-poly(ethylene glycol)-azide, an propargyl, an aminooxy-poly(ethylene glycol)-DBCO, an aminooxy-poly(ethylene glycol)-bicyclononyne (BCN), a cyanine 555 aminooxy, a cyanine 647 aminooxy, an aminooxy-biotin, a naphthalene-containing aminooxy-fluorescent dye, a guanidine-containing aminooxy-fluorescent dye, a Cy5-PEG-aminooxy, or a fluorescent dye hydrazide.

The present disclosure also provides a method for 5′-end- and 3′-end-labeling of a nucleic acid, comprising providing a target nucleic acid to be labeled, adding to the target nucleic acid a 5′-end glycosylase and a nucleotide having a reactive moiety; creating an intermediate nucleic acid having an abasic site at a 5′-end of the target nucleic acid and incorporating the nucleotide having the reactive moiety to the 3′-end of the intermediate nucleic acid; providing an aldehyde-reactive compound carrying a detectable label for coupling with the intermediate nucleic acid at the abasic site at the 5′-end and a desired molecule having a corresponding functional moiety capable of reacting with the reactive moiety; and exposing the intermediate nucleic acid to the aldehyde-reactive compound carrying a detectable label and to the desired molecule having a corresponding functional moiety, thereby forming a labeled nucleic acid with the detectable label coupling with the intermediate nucleic acid at the abasic site to form a labeled nucleic acid with the detectable label attached at the 5′-end and with a linkage forming between the reactive moiety and the corresponding functional moiety at the 3′-end.

The present disclosure also provides a kit for 5′-end- and 3′-end-labeling of a nucleic acid, and the kit comprises a 5′-end glycosylase, an aldehyde-reactive compound, a nucleotide having a reactive moiety, a polymerase to incorporate the nucleotide having the reactive moiety to the 3′-end of the nucleic acid, and a desired molecule having a corresponding functional moiety capable of reacting with the reactive moiety.

### BRIEF DESCRIPTION OF THE DRAWINGS

The present disclosure will become more readily appreciated and better understood by reference to the following descriptions, when taken in conjunction with the accompanying drawings.

FIG. 1 is a schematic diagram depicting an exemplary method of introducing a 3′-modification to a nucleic acid or polynucleotide and the enrichment of the labeled nucleic acid or polynucleotide.

FIGS. 2A and 2B show an example of labeling a Cy5-fluorescent dye/fluorophore to the 3′-end of a polynucleotide via the enzymatic synthesis of 3′-O-azidomethyl deoxynucleotide (3′-AZ-dNTP) to the 3′-end of the polynucleotide, followed by the azide-alkyne click conjugation reaction between the incorporated 3′-O-azidomethyl deoxynucleoside monophosphate (3′-AZ-dNMP) and the alkyne-modified Cy5-fluorescent dye moiety. FIGS. 2A and 2B are images from the same gel. While FIG. 2A depicts the gel electrophoresis result of the unlabeled and labeled polynucleotides visualized by staining with SYBR Gold dye, FIG. 2B illustrates the electrophoretic location of nucleic acids labeled with the Cy5-fluorescent dye after the enrichment step. Lane 1: the electrophoretic location of the target polynucleotide (45-mer single-stranded DNA); lane 2: the electrophoretic location of the target polynucleotide plus an incorporated 3′-AZ-dNMP at the 3′-end; and lane 3: the electrophoretic location of the polynucleotide plus an incorporated 3′AZ-dNMP coupled with a Cy5-fluorescent dye at the 3′-end after the enrichment step.

FIG. 3 shows an example of labeling a fluorescent quencher (BHQ1) to the 3′-end of a polynucleotide via the enzymatic synthesis of 3′-O-azidomethyl deoxynucleotide (3′-AZ-dNTP) to the 3′-end of the polynucleotide, followed by the azide-alkyne click conjugation reaction between the incorporated 3′-AZ-dNMP and the alkyne modified fluorescent quencher moiety. Lane 1: the electrophoretic location of the polynucleotide (45-mer single-stranded DNA); lane 2: the electrophoretic location of the polynucleotide plus an incorporated 3′-AZ-dNMP at the 3′-end; lane 3: the electrophoretic location of the polynucleotide plus an incorporated 3′-AZ-dNMP coupled with, or without, a fluorescent quencher at the 3′-end; and lane 4: the electrophoretic location of the polynucleotide plus an incorporated 3′-AZ-dNMP coupled with a fluorescent quencher at the 3′-end after the enrichment step.

FIGS. 4A and 4B show an example of labeling a Cy5-fluorescent dye/fluorophore to the 3′-end of a polynucleotide via the enzymatic synthesis of 3′-O-azidomethyl deoxynucleotide (3′-AZ-dNTP) to the 3′-end of the polynucleotide, followed by the azide-DBCO conjugation reaction between the incorporated 3′-AZ-dNMP and the DBCO-modified Cy5-fluorescent dye moiety. FIGS. 4A and 4B are images from the same gel. While FIG. 4A depicts the gel electrophoresis result of the unlabeled and labeled polynucleotides visualized by staining with the SYBR Gold dye, FIG. 4B illustrates the electrophoretic location of nucleic acids labeled with the Cy5-fluorescent dye at the 3′-end. Lane 1: the electrophoretic location of the target polynucleotide (45-mer single-stranded DNA); and lane 2: the electrophoretic location of the polynucleotide plus an incorporated 3′-AZ-dNMP conjugated with a Cy5-fluorescent dye at the 3′-end.

FIG. 5 shows an example of labeling a fluorescent quencher (BHQ1) to the 3′-end of a target polynucleotide via the enzymatic synthesis of 3′-O-azidomethyl deoxynucleotide (3′-AZ-dNTP) to the 3′-end of the polynucleotide, followed by the azide-DBCO conjugation reaction between the incorporated 3′-AZ-dNMP and the DBCO-modified fluorescent quencher moiety. In this figure, lane 1 shows the electrophoretic location of the polynucleotide (45-mer single-stranded DNA); lane 2 shows the electrophoretic location of the target polynucleotide plus an incorporated 3′-AZ-dNMP at the 3′-end; lane 3 shows the electrophoretic location of the target polynucleotide plus an incorporated 3′-AZ-dNMP coupled with, or without, a fluorescent quencher at the 3′-end; and lane 4 shows the electrophoretic location of the target polynucleotide plus an incorporated 3′-AZ-dNMP coupled with a fluorescent quencher at the 3′-end after the enrichment step.

FIG. 6 shows the electrophoretic result of an example of labeling the 3′-end of a polynucleotide having a partial double-stranded region formed by a 60-mer strand and a 20-mer strand. In FIG. 6, lane S shows the electrophoretic location of the partial double-stranded polynucleotide before a labeling reaction; lane 1 shows the result of formation of a 61-mer forward strand carrying an azide group at the 3′-end; lane 2 shows the result after the labeling reaction; and lane 3 shows the result after a clean-up reaction.

FIG. 7 shows the electrophoretic result of labeling a Cy5-fluorescent dye/fluorophore to the 3′-end of a polynucleotide through the enzymatic synthesis of different 3′-O-azidomethyl deoxynucleotides. Lane S1: the electrophoretic location of the polynucleotide before incorporating Cy5-labeled 3′-AZ-dATP; lane 1: the electrophoretic location of the Cy5-labeled 3′-AZ-dATP-incorporated polynucleotide after a labeling reaction; lane 1-1: the electrophoretic location of Cy5-labeled polynucleotide after a clean-up reaction; lane S2: the electrophoretic location of the polynucleotide before incorporating Cy5-labeled 3′-AZ-dGTP; lane 2: the electrophoretic location of the Cy5-labeled 3′-AZ-dGTP-incorporated polynucleotide after a labeling reaction; lane 2-1: the electrophoretic location of Cy5-labeled polynucleotide after a clean-up reaction; lane S3: the electrophoretic location of the polynucleotide before incorporating IF700-labeled 3′-AZ-dCTP; lane 3: the electrophoretic location of the IF700-labeled 3′-AZ-dCTP-incorporated polynucleotide after the labeling reaction; and lane 3-1: the electrophoretic location of IF700-labeled polynucleotide after a clean-up reaction.

FIG. 8 shows the Urea-PAGE result of 5′-end labeling on a single-stranded DNA (ssDNA) and a duplex DNA with a uracil-DNA glycosylase derived from Micrococcus luteus (MluUDG) and an aldehyde-reactive probe (ARP). “S” denotes the lane containing only unlabeled DNA.

FIG. 9 shows the Urea-PAGE result of 5′-end labeling on a single stranded DNA and a duplex DNA with a uracil-DNA glycosylase derived from Micrococcus luteus (MluUDG) and an aminooxy-5(6)-FAM. “S” denotes the lane containing only unlabeled DNA.

FIG. 10 shows the Urea-PAGE result of 5′-end labeling on a 5′-phosphorylated single-stranded DNA and a duplex DNA with a uracil-DNA glycosylase derived from Micrococcus luteus (MluUDG) and a naphthalene- and guanidine-containing aminooxy-FAM. “S” denotes the lane containing only unlabeled DNA.

FIG. 11 shows the Urea-PAGE result of 5′-end labeling on a single-stranded DNA with a uracil-DNA glycosylase derived from Micrococcus luteus (MluUDG) and an aldehyde-reactive probe (ARP) followed with a cleanup step to eliminate the unlabeled DNA using a phage lambda exonuclease (lambda exo). “S” denotes the lane containing only unlabeled DNA.

FIG. 12 shows the Urea-PAGE result of 5′-end labeling on a single-stranded DNA and a duplex DNA with a uracil-DNA glycosylase derived from Micrococcus luteus (MluUDG) and a naphthalene- and guanidine-containing aminooxy-FAM followed with a clean-up step to eliminate the unlabeled DNA using a phage lambda exonuclease (lambda exo). “S” denotes the lane containing only unlabeled DNA.

FIG. 13 shows the electrophoresis results of 5′-end labeling, 3′-end labeling, and dual labeling of a double-stranded DNA. Lane 1 shows the electrophoretic location of a 21-mer uracil-containing ssDNA; lane 2 shows the electrophoretic location of 5′-end FAM-labeled product; lane 3 shows the electrophoretic location of the 3′-end labeled product; and lane 4 shows the electrophoretic location of dual-labeled products.

FIG. 14 shows the electrophoresis results of 3′-end labeling and dual labeling of a double-stranded DNA. Lanes 1 and 2 show the electrophoretic locations of a 26-mer uracil-containing ssDNA and a 27-mer complementary strand, respectively; lane 3 shows the electrophoretic location of the 3′-end labeled product; lane 4 shows the electrophoretic location of 3′-end labeled product after exonuclease digestion to remove unlabeled DNAs; lane 5 shows the electrophoretic location of the dual-labeled products; and lane 6 shows the electrophoretic location of the dual-labeled products after exonuclease digestion to remove unlabeled DNAs.

FIG. 15 shows the electrophoresis results of sequential or simultaneous dual-labeling of a double-stranded DNA. Lanes 1 and 2 show the electrophoretic locations of a 25-mer uracil-containing ssDNA and a 26-mer complementary strand, respectively; lane 3 shows the electrophoretic location of sequential dual labeling products of a double-stranded DNA after exonuclease digestion; lane 4 shows the electrophoretic location of simultaneous dual labeling products of a double-stranded DNA after exonuclease digestion; lane 5 shows the electrophoretic location of sequential dual labeling products of a double-stranded DNA with azide-DBCO ligation at 3′-end after exonuclease digestion; and lane 6 shows the electrophoretic location of simultaneous dual labeling products of a double-stranded DNA with azide-DBCO ligation at 3′-end after exonuclease digestion.

FIG. 16A shows Scheme 1 for the preparation of a 3′-azide-labeled target polynucleotide containing a 3′-O-azidomethyl group.

FIG. 16B shows Scheme 2 for the labeling of a Cy5 fluorophore to the 3′-end of a target polynucleotide.

FIG. 16C shows Scheme 3 for the labeling of a fluorescent quencher to the 3′-end of a target polynucleotide.

FIG. 16D shows Scheme 4 for the labeling of a Cy5 fluorophore to the 3′-end of a target polynucleotide.

FIG. 16E shows Scheme 5 for the labeling of a fluorescent quencher to the 3′-end of a target polynucleotide.

### DETAILED DESCRIPTION

All terms including descriptive or technical terms which are used herein should be construed as having meanings that are obvious to one of ordinary skill in the art. However, the terms may have different meanings according to an intention of one of ordinary skill in the art, case precedents, or the appearance of new technologies. Also, some terms may be arbitrarily selected by the applicant, and in this case, the meaning of the selected terms will be described in detail in the descriptions of the present disclosure. Thus, the terms used herein are defined based on the meaning of the terms together with the descriptions throughout the specification.

The practice of the present disclosure employs, unless otherwise indicated, conventional techniques of molecular biology, microbiology, cell biology, biochemistry, and immunology, which are well within the purview of a skilled artisan in the art. Such techniques are explained fully in the literature, such as “Molecular Cloning: A Laboratory Manual,” second edition (Sambrook, et al., 1989), Cold Spring Harbor Press; “Oligonucleotide Synthesis” (M. J. Gait, 1984); “Methods in Molecular Biology,” Humana Press; “Cell Biology: A Laboratory Notebook” (J. E. Cellis, ed., 1998) Academic Press; “Animal Cell Culture” (R. I. Freshney, ed., 1987); “Handbook of Experimental Immunology” (Weir, 1996); “Introduction to Cell and Tissue Culture” (J. P. Mather and P. E. Roberts, 1998); “Cell and Tissue Culture: Laboratory Procedures” (A. Doyle, J. B. Griffiths, and D. G. Newell, eds., 1993-8); “Methods in Enzymology” (Academic Press, Inc.); “Handbook of Experimental Immunology” (D. M. Weir and C. C. Blackwell, eds.); “Gene Transfer Vectors for Mammalian Cells” (J. M. Miller and M. P. Calos, eds., 1987); “Current Protocols in Molecular Biology” (F. M. Ausubel, et al., eds., 1987); “PCR: The Polymerase Chain Reaction (Mullis, et al., eds., 1994); and “Current Protocols in Immunology” (J. E. Coligan et al., eds., 1991); “Short Protocols in Molecular Biology” (Wiley and Sons, 1999); “Immunobiology” (C. A. Janeway and P. Travers, 1997); “Antibodies” (P. Finch, 1997); “Antibodies: a practical approach” (D. Catty., ed., IRL Press, 1988-1989); “Monoclonal antibodies: a practical approach” (P. Shepherd and C. Dean, eds., Oxford University Press, 2000); “Using antibodies: a laboratory manual” (E. Harlow and D. Lane (Cold Spring Harbor Laboratory Press, 1999); The Antibodies (M. Zanetti and J. D. Capra, eds., Harwood Academic Publishers, 1995). Particularly useful techniques for particular embodiments will be discussed in the sections that follow. Without further elaboration, it is believed that one skilled in the art can, based on the above descriptions, utilize the present disclosure to its fullest extent. The following embodiments are, therefore, to be construed as merely illustrative, and not limitative of the remainder of the disclosure in any way whatsoever. All publications cited herein are incorporated by reference for the purposes or subject matter referenced herein.

As used herein, the singular forms “a,” “an,” and “the” are intended to include the plural forms, unless the context clearly indicates otherwise. The terms “includes,” “including,” “comprises,” and “comprising” are used in either the detailed descriptions and/or the claims, and such terms are intended to be inclusive in a manner of not excluding others, such as other components, materials, steps, etc. The terms “sec,” “min,” and “hr” as used herein are abbreviations of “second,” “minute,” and “hour.”

Where ranges are given, endpoints are included. Furthermore, unless otherwise indicated or otherwise evident from the context and understanding of one of ordinary skill in the art, values that are expressed as ranges can assume any specific value or sub-range within the stated ranges in different embodiments of this disclosure, unless the context clearly dictates otherwise.

As used herein, the terms “about,” “approximately,” and “around” generally mean within 10%, 5%, 1%, or 0.5% of a given value or range. Alternatively, the term “about,” “approximately,” and “around” mean within an acceptable standard error of the mean when considered by one of ordinary skill in the art. Unless otherwise expressly specified, all of the numerical ranges, amounts, values, and percentages such as those for quantities of materials, durations of time periods, temperatures, operating conditions, ratios of amounts, and the likes disclosed herein should be understood as modified in all instances by the terms “about,” “approximately,” or “around.”

As used herein, the term “derived,” when referring to a biological sample, indicates the sample being obtained from the stated source at some point in time. For example, a biological sample derived from an organism can represent a primary biological sample obtained directly from the organism (i.e., unmodified), or can be modified, e.g., by introduction of a recombinant vector, by culturing under particular conditions, or immortalization.

As used herein, the phrase “at least one,” in reference to a list of one or more elements, should be understood to mean at least one element selected from any one or more of the elements in the list of elements, but not necessarily including at least one of each and every element listed within the list of elements and not excluding any combinations of elements in the list of elements. This definition also allows that elements may optionally be present other than the elements identified within the list of elements to which the phrase “at least one” refers, whether related or unrelated to those elements identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements).

As used herein, an abasic site, also known as an apurinic/apyrimidinic (AP) site, encompasses any chemical structure following removal of a base portion (including the entire base) with an agent capable of cleaving a base portion of a nucleotide, e.g., by the treatment of a nucleotide (present in a polynucleotide chain) with an agent (e.g., an enzyme, an acidic condition, or a chemical reagent) capable of effecting cleavage of a base portion of a nucleotide. In an embodiment, an AP site is a position in the backbone of nucleic acids such as deoxyribonucleic acids (DNA) or ribonucleic acids (RNA) that lacks a nucleobase, i.e., a deoxyribose of the DNA backbone or a ribose of the RNA backbone not covalently linked to either a purine base, such as adenine (A) or guanine (G), or a pyrimidine base, such as cytosine (C), uracil (U) or thymine (T). An AP site may be internal within the nucleotide sequence of the nucleic acids at both 5′- and 3′-ends of the nucleic acids, or at one end of the nucleic acids, such as the 5′-end or the 3′-end.

The terms “nucleic acid,” “nucleic acid sequence,” and “nucleic acid fragment” as used herein refer to a nucleotide sequence in a single-stranded or double-stranded form, of which the sources are not limited herein, and generally, include naturally occurring nucleotides or artificial chemical mimics. The term “nucleotide” as used herein refers to the monomeric unit of nucleic acids or polynucleotides as described hereafter, having a glycoside with or without a nucleobase, and one or more internucleotide linkages, e.g., phosphodiester linkage. In some embodiments, the nucleobase includes naturally occurring bases such as adenine (A), thymine (T), cytosine (C), guanine (G), and uracil (U), non-naturally occurring bases such as xanthine, hypoxanthine, isoguanine, and isocytosine, as well as any analogs or derivatives thereof. In some embodiments, a nucleotide with an abasic site (loss of nucleobase) is also included within the scope of the present disclosure. In some embodiments, the sugars in the glycoside include naturally occurring sugars such as pentose sugars (e.g., deoxyribose and ribose), non-naturally occurring sugars, and the analogs thereof. In some embodiments, nucleotides are linked via internucleotide linkages such as, but not limited to, phosphate, boranephosphate, phosphorothioate, phosphodiester, phosphotriester, H-phosphonate, aminophosphonate, methylphosphonate, phosphonoacetate, sulfur phosphonoacetate, or other variants of the phosphate backbone of natural nucleic acids. The term “nucleotide” as used herein also encompasses structural analogs in place of natural or nonnatural nucleotides, such as modified nucleotides. For example, the term “xeno nucleotide” refers to the nucleotide being modified to have a different sugar moiety than those contained in a natural DNA or RNA. The exemplary nucleic acids having the xeno nucleotide, i.e., xeno-nucleic acids (XNA), include but not limited to peptide nucleic acid (PNA), locked nucleic acid (LNA), 1,5-anhydrohexitol nucleic acid (HNA), threose nucleic acid (TNA), glycol nucleic acid (GNA), cyclohexene nucleic acid (CeNA), and FANA (fluoro arabino nucleic acid).

As used herein, the term “polynucleotide” refers to a polymer of nucleotides and is generic to any type of nucleic acids such as natural or non-natural DNA or RNA and modified nucleic acids such as xeno-nucleic acids (XNA) as described herein. A polynucleotide can also include any combinations of glycosides with or without a nucleobase and internucleotide linkages. Unless otherwise specified, the polynucleotide featured herein has an intrinsic directionality in terms of the 5′-end of one nucleotide to the 3′-end of its neighboring nucleotide, where the template-independent synthesis of a polynucleotide provided herein proceeds in a 5′ to 3′ direction.

The term “polynucleotide” used herein is not intended to be distinct in length of nucleotide unit, where the term refers only to the polymeric molecule structure. That is to say, a polynucleotide used herein is interchangeable with the term “oligonucleotide,” and can range in size from a few monomeric nucleotide units to several thousands of monomeric nucleotide units, such as 2 to 5 nucleotides, 5 to 20 nucleotides, 20 to 100 nucleotides, 100 to 1,000 nucleotides, or longer. A polynucleotide can be composed entirely of natural or non-natural occurring, modified or non-modified deoxyribonucleotides, entirely of natural or non-natural occurring, modified or nonmodified ribonucleotides, or chimeric mixtures thereof. Nucleobases (also known as nitrogenous bases) contained in a polynucleotide may be, for example, adenine, thymine, cytosine, guanine, uracil, xanthine, hypoxanthine, isocytosine, or isoguanine. In addition, a polynucleotide may contain one or more abasic sites (apurinic/apyrimidinic site), also known as AP sites.

The terms “nucleic acids” and “polynucleotides” may be used interchangeably herein to refer to deoxyribonucleotides or ribonucleotides and polymers thereof in either single- or double-stranded form. These terms encompass nucleic acids containing known nucleotide analogs or modified backbone residues or linkages, which are synthetic, naturally occurring, and non-naturally occurring, and/or which have similar chemical properties as the reference nucleic acids, and/or which are metabolized in a manner similar to the reference nucleotides. Unless otherwise indicated, a particular nucleic acid sequence also encompasses conservatively modified variants thereof (e.g., degenerate codon substitutions) and complementary sequences, as well as the sequence explicitly indicated. In some embodiments, nucleotides are linked via internucleotide linkages such as, but not limited to, phosphate, boranephosphate, phosphorothioate, phosphodiester, phosphotriester, H-phosphonate, aminophosphonate, methylphosphonate, phosphonoacetate, sulfur phosphonoacetate, or other variants of the phosphate backbone of natural nucleic acids. The term “nucleotide” as used herein also encompasses structural analogs in place of natural or nonnatural nucleotides, such as modified nucleotides. For example, the term “xeno nucleotide” refers to the nucleotide being modified to have a different sugar moiety than those contained in a natural DNA or RNA. The exemplary nucleic acids having the xeno nucleotide, i.e., xeno-nucleic acids (XNA), include, but are not limited to, peptide nucleic acid (PNA), locked nucleic acid (LNA), 1,5-anhydrohexitol nucleic acid (HNA), threose nucleic acid (TNA), glycol nucleic acid (GNA), cyclohexene nucleic acid (CeNA), and fluoro-arabino nucleic acid (FANA).

The nucleic acids as used herein may be 5 bases to 10,000 bases in length, such as 10 to 3,000 bases in length, 10 to 1,000 bases in length, or 10 to 100 bases in length. The nucleic acids isolated from biological sources may be greater than 1,000 bases in length and may be fragmented, for example, by sonication, for use as described herein.

In some embodiments, the nucleic acids to be labeled by the method of the present disclosure can be about 10, about 15, about 20, about 25, about 30, about 35, about 40, about 50, about 65, about 75, about 85, about 100, about 125, about 150, about 175, about 200, about 225, about 250, about 300, about 350, about 400, about 450, about 500, about 550, about 600, about 650, or more nucleotides in length. In some embodiments, said nucleic acids can be at least about 5, about 6, about 7, about 8, about 9, about 10, about 15, about 20, about 25, about 30, about 35, about 40, about 50, about 65, about 75, about 85, about 100, about 125, about 150, about 175, about 200, about 225, about 250, about 300, about 350, about 400, about 450, about 500, about 550, about 600, about 650 or more nucleotides in length. In other embodiments, said nucleic acids can be less than about 20, about 25, about 30, about 35, about 40, about 50, about 65, about 75, about 85, about 100, about 125, about 150, about 175, about 200, about 225, about 250, about 300, about 350, about 400, about 450, about 500, about 550, about 600, or about 650 nucleotides in length. It is understood that the lengths of nucleic acids may represent an average size in the population.

As used herein, a glycosylase is an enzyme capable of excising a base portion of a nucleotide and creating an AP site in a nucleic acid, which includes N-glycosylases and is also called as “DNA glycosylase” or “glycosidase,” including, but not limited to, uracil N-glycosylase (UNG) that specifically cleaves dUTP and is interchangeably termed as “uracil DNA glycosylase” (UDG); hypoxanthine-N-glycosylase; hydroxymethyl cytosine-N-glycosylase; 3-methyladenine DNA glycosylase; 3- or 7-methylguanine DNA glycosylase; hydroxymethyl uracil DNA glycosylase; and T4 endonuclease V. A glycosylase cleaves a base portion of the nucleotide in the middle, or at either or both ends of a nucleic acid. As used herein, a 5′-end glycosylase excises a base portion of the nucleotide at the 5′-end of a nucleic acid.

The term “initiator” as used herein refers to a nucleoside monomer, a nucleotide monomer, an oligonucleotide, a polynucleotide, or modified analogues thereof, from which a nucleic acid is to be synthesized by a nucleic acid polymerase de novo. The term “initiator” may also refer to an XNA having a 3′-hydroxyl group, such as a 3′-hydroxyl-PNA.

According to the present disclosure, the initiator may also be linked or immobilized to a solid support, and a linking nucleotide is coupled to a 3′-terminal nucleotide of the initiator and a 5′-terminal nucleotide of the synthesized nucleic acid. The initiator may be directly attached to the solid support, attached to the support via a linker, or immobilized via physical interactions such as adsorption, electrostatic interaction, and hydrogen bonds. Examples of the solid support include, but are not limited to, microarrays, beads (coated or non-coated), columns, optical fibers, wipes, nitrocellulose, nylon, glass, quartz, diazotized membranes (paper or nylon), silicones, polyformaldehyde, cellulose, cellulose acetate, paper, ceramics, metals, metalloids, semiconductive materials, magnetic particles, plastics (such as polyethylene, polypropylene, and polystyrene), gel forming materials (such as proteins (e.g., gelatins), lipopolysaccharides, silicates, agarose, polyacrylamides, or methyl methacrylate polymers), sol-gels, porous polymers, hydrogels, nanostructured surface nanotubes (such as carbon nanotubes), and nanoparticles (such as gold nanoparticles or quantum dots).

The term “polymerase” as used herein refers to an enzyme/protein capable of synthesizing nucleic acids, which is generically a DNA polymerase, an RNA polymerase, or a functionally equivalent enzyme, including naturally-occurring enzymes, modified enzymes, enzyme subunits and the derivatives thereof. For example, an amino acid sequence modification (e.g., mutation and functional group substitution) can be applied to these enzymes for desired purposes, such as removing the 5′ to 3′ exonuclease activity and enhancing the polymerase activity, to obtain altered enzymes with improved properties, such as thermostability/thermotolerance and catalytic efficiency.

According to the present disclosure, the term “polymerase” may be a template dependent polymerase or a template-independent polymerase. The polymerase may include a family-A DNA polymerase (e.g., T7 DNA polymerase, Pol I, Pol γ, θ, and v), a family-B DNA polymerase (e.g., Pol II, Pol B, Pol ζ, Pol α, δ and ε), a family-C DNA polymerase (e.g., Pol III), a family-D DNA polymerase (e.g., PolD), a family-X DNA polymerase (e.g., Pol β, Pol σ, Pol λ, Pol μ and terminal deoxynucleotidyl transferase), a family-Y DNA polymerase (e.g., Pol Λ, Pol κ, Pol η, DinB, Pol IV and Pol V), a reverse transcriptase (e.g., telomerase and hepatitis B virus), and enzymatically active fragments thereof.

Non-limiting examples of widely employed template-dependent polymerases include T7 DNA polymerase of T7 bacteriophage and T3 DNA polymerase of T3 bacteriophage, which are DNA-dependent DNA polymerases; T7 RNA polymerase of T7 bacteriophage and T3 RNA polymerase of T3 bacteriophage, which are DNA-dependent RNA polymerases; DNA polymerase I or its fragment known as the Klenow fragment of Escherichia coli, which is a DNA-dependent DNA polymerase; Thermophilus aquaticus DNA polymerase, Tth DNA polymerase and Vent DNA polymerase, which are thermostable DNA-dependent DNA polymerases; eukaryotic DNA polymerase β, which is a DNA-dependent DNA polymerase; telomerase, which is an RNA-dependent DNA polymerase; and non-protein catalytic molecules, such as modified RNA (ribozymes; Unrau & Bartel, 1998) and DNA with template-dependent polymerase activity.

Non-limiting examples of the template-independent polymerases include reverse transcriptase, poly A polymerase, DNA polymerase theta (0), terminal deoxynucleotidyl transferase (TdT), and DNA polymerase mu (u). Since polymerases suitable for performing nucleic acid synthesis, nucleotide addition/incorporation, and process of nucleic acid synthesis are within the expertise and routine skills of those skilled in the art, further details thereof are omitted herein for the sake of brevity. Furthermore, the B-family DNA polymerases provided previously by inventors are also suitable for being used under template-independent conditions, and the U.S. Pat. No. 11,591,629B2 are hereby incorporated entirely by reference.

The term “modification” as used herein refers to the alteration(s) of the chemical structure of a reactant molecule. When a nucleic acid is used as a reactant molecule, the means of modifications include, but are not limited to, the introduction of an additional chemical group/moiety to the nucleic acid, removal or substitution of an original chemical group/moiety from the nucleic acid, or the combination thereof, regardless of the source of the nucleic acid. Alternatively, the modification(s) may be introduced to a specific sequence of nucleic acids during the de novo nucleic acid synthesis resulting in a direct modification, or modifications, on the nucleic acid. For example, a fluorophore-labeled nucleotide analogue can be incorporated into a nucleic acid alongside with natural counterparts to become a “fluorescent labeled” nucleic acid. Likewise, a site-specific modification, or modifications, can also be inserted enzymatically into a nucleic acid by incorporating nucleotide(s) carrying desired modification(s). For example, a nucleoside triphosphate having a 3′-O-azidomethyl group can be enzymatically introduced to the 3′-end of a nucleic acid, and thus, directly adds an azidomethyl modification to the 3′-end of the nucleic acid. Such modifications result in the addition of a nucleotide together with a site-specific chemical group to a target nucleic acid.

As used herein, the term “detecting” or “detection” refers to both quantitative and qualitative determinations and as such, the term “detecting” or “detection” is used interchangeably herein with “assaying,” “measuring,” and the like. Where a quantitative determination is intended, the phrase “determining an amount” and the like is used. Where either a qualitative or quantitative determination is intended, the phrase “determining a level” or “detecting a level” may be used.

As used herein, the term “exonuclease” refers to any wild-type or variant enzyme, which is capable of cleaving phosphodiester bond(s) linking the end nucleotides of an oligonucleotide or a polynucleotide, such as a 5′ to 3′ exonuclease, a 3′ to 5′ exonuclease, and a poly(A)-specific 3′ to 5′ exonuclease. Non-limiting examples of exonucleases include exonuclease I, exonuclease II, exonuclease III, exonuclease IV, exonuclease V, exonuclease VI, exonuclease VII, exonuclease VII, Xml, and Rat1.

As used herein, the term “5′ to 3′ exonuclease” refers to an exonuclease that breaks phosphodiester bonds at the 5′ end of an oligonucleotide or a polynucleotide. Non-limiting examples of 5′ to 3′ exonucleases include T5 exonuclease, T7 exonuclease, bacterial alkaline exonuclease, viral alkaline exonuclease, phage lambda exonuclease, 5′-exonuclease of DNA polymerase I, exonuclease VIII, RecJ, RecJf, Tth RecJ, Mpn NrnA, human exonuclease 5, human exonuclease 1, SNM1, SNM1A, human SNM1B/Apollo, bovine SNM1B, SXT-Exo, phospholipase D3, phospholipase D4, Sso1391-Csa1, Sto0027-Csa1, Ttx1248-Csa1, Sso1451-Csa1, Sto2633-Csa1, Pfu1793-Cas4, Sto2501, Sso0001, Sto2331-Cas4, Ttx 1245-Cas4, Sso1449-Cas4, Sto2635-Cas4, Sso1392-Cas4, SIRV2 gp19, and bacterial AddB.

As used herein, the term “3′-end” generally refers to a region or position in a polynucleotide or oligonucleotide downstream from the 5′-region or position in the same polynucleotide or oligonucleotide.

As used herein, the term “5′-end” generally refers to a region or position in a polynucleotide or oligonucleotide upstream from the 3′-region or position in the same polynucleotide or oligonucleotide.

As used herein, an aldehyde-reactive compound is a class of compounds that reacts to or forms a bond with an aldehyde group. In an embodiment, an aldehyde-reactive compound structurally is a compound having at least one primary amine, a hydrazide, an acylhydrazide, a compound having an aminooxy (—ONH₂) group, a compound having a naphthalene-containing aminooxy group and/or a guanidine-containing aminooxy group.

As used herein, the term “label” (interchangeably called a “detectable label”) refers to a chemical group or functional moiety that is associated or linked with a polynucleotide (interchangeably called “labeling”). The labeled polynucleotide may be directly or indirectly detected, generally through a detectable signal. The detectable label can be attached (or associated) either directly or through a non-interfering linkage group with other moieties capable of specifically associating with one or more sites to be labeled. The detectable label may be covalently or non-covalently associated as well as directly or indirectly associated.

As used herein, the term “mono-functional DNA glycosylase” refers to a naturally existing mono-functional glycosylase that intrinsically contains only a DNA glycosylase activity. The term “mono-functional DNA glycosylase” may also refer to a mono-functional glycosylase that is derived from a bi-functional DNA glycosylase naturally having both DNA glycosylase and abasic-site lyase (AP lyase) activities by eliminating or inactivating the AP lyase domain of the bi-functional DNA glycosylase.

As used herein, the term “enzymatically active fragment” refers to a fragment of a catalytically or enzymatically active protein or polypeptide which contains at least 10%, e.g., at least 20%, at least 30%, at least 40%, at least 50%, at least 60%, at least 70%, at least 80%, at least 90%, or at least 95% of activity of the protein or polypeptide from which the fragment is derived.

The processes of signal detection are known in the art. Signal detection may be visual or utilize a suitable instrument appropriate for the label used, such as a spectrometer, fluorimeter, luminometer, phosphorimager, Geiger counter, scintillation counter, or microscope. For example, where the label is a radioisotope, detection can be achieved by using, for example, a scintillation counter, or photographic film as in autoradiography. Where a fluorescent label is used, detection may be achieved by exciting the fluorochrome with an appropriate wavelength of light and detecting the emitting fluorescence, such as by a fluorescence microscopy, visual inspection, photographic film, fluorometer, luminometer, charge-coupled device (CCD) cameras, and scanner. Where enzymatic labels are used, detection may be achieved by providing appropriate substrates for the enzyme and detecting the resulting reaction product. For example, many substrates of horseradish peroxidase, such as o-phenylenediamine, give colored products. Instruments suitable for high sensitivity detection are known in the art. Otherwise, the signal amplification strategies can be optionally used to facilitate the detection of low-abundance molecular targets.

Although the present disclosure is illustrated by specific embodiments and optional features, it is understood that modifications and variations of the concepts herein disclosed may be resorted to by those skilled in the art, and that such modifications and variations are considered to be within the scope of the present disclosure.

### Example

Exemplary embodiments according to the present disclosure are further described in the following examples, which should not be construed to limit the scope of the present disclosure. The materials and methods used in the following Examples are described in detail below. The materials used in the present disclosure but unannotated herein are commercially available.

Provided herein are examples of modifying nucleic acids using the method of the present disclosure, and several exemplary modifications of polynucleotides are demonstrated to produce a labeled nucleic acid with a fluorophore or quencher at its 3′-end.

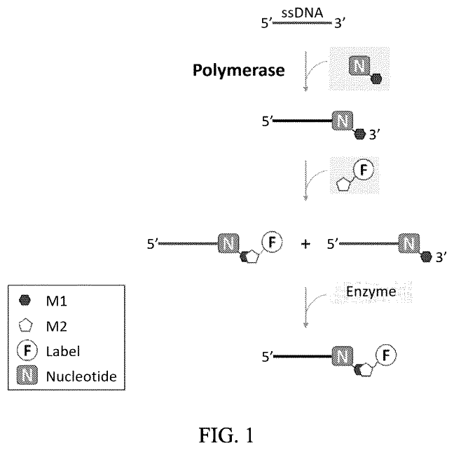

As illustrated in FIG. 1, the methods used in these modifications can be practically divided into three sequential steps: 1) using a polymerase to incorporate a modified nucleotide carrying a reactive moiety to the 3′-end of a target polynucleotide; 2) conjugating a desired molecule having a label moiety such as a fluorescent dye or a quencher and a corresponding functional moiety to the 3′-end of the target polynucleotide; 3) degrading unlabeled or incompletely labeled polynucleotides by a 3′ to 5′ exonuclease to enrich the desired, labeled polynucleotide.

In some embodiments, an enzymatic synthesis approach is used to introduce the reactive moiety to the target polynucleotide. For example, a nucleotide analogue, or analogues, is enzymatically added to the 3′-hydroxyl (3′-OH) end of a single-stranded, nucleic acid initiator (the target polynucleotide) in a template-independent synthesis manner to produce a polynucleotide with a desired reactive moiety. In at least one embodiment, the desired reactive moiety is an azide (N3) or azido group, and a suitable reagent/compound, such as a nucleotide analogue, containing an azido moiety, such as a 3′-O-azidomethyl group, may be used to introduce such a modification to the 3′-end of polynucleotides. The following examples are further provided based on this scenario.

### Example 1: Preparation of a Polynucleotide Containing a 3′-O-azidomethyl Group

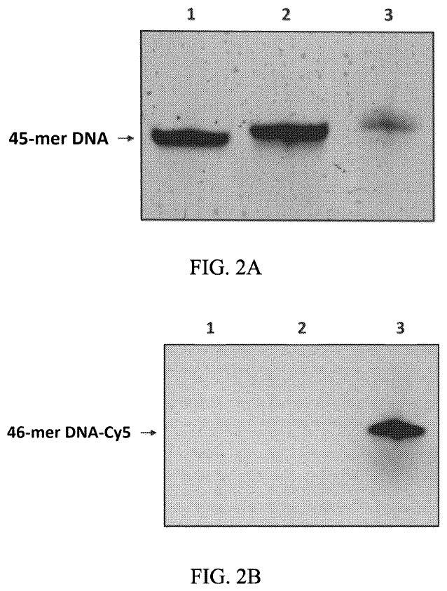

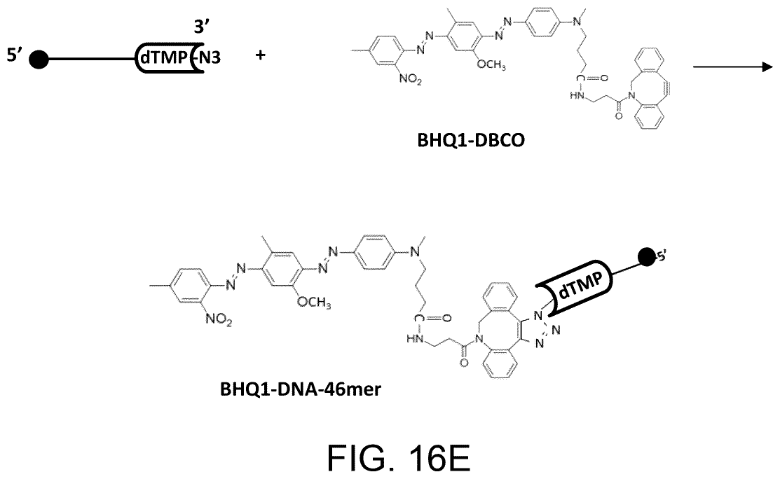

A 45-mer DNA polynucleotide containing a fluorescein label at the 5′-end (5′-FAM-45-mer DNA) was used as the target polynucleotide for 3′-modification or labeling. To introduce an azidomethyl group to the 3′-end of the 5′-FAM-45-mer DNA, the 3′-O-azidomethyl-deoxynucleoside triphosphate (3′-AZ-dNTP) was used as a template-independent DNA synthesis substrate for the selective B-family DNA polymerase or its variant. The exemplary polymerase used herein may be an in-house polymerase variant derived from Vent DNA polymerase, which can efficiently incorporate the 3′-AZ-dNTP to the 3′-end of the target polynucleotide. The nucleotide incorporation reaction was performed in the reaction mixture (10 μL) containing 100 nM of 5′-FAM-45-mer DNA target polynucleotide, 0.25 mM manganese chloride (MnCl₂), and 200 nM of polymerase. The reaction was initiated by the addition of 25 μM of 3′-O-azidomethyl-dTTP (3′-AZ-dTTP) and then incubated at 60° C. for a designated period of time. Time periods from 2 minutes to 30 minutes have been used. The reaction was then terminated by adding a 10 μL of 2× quench solution (95% de-ionized formamide and 25 mM EDTA). The reaction mixture was further denatured at 95° C. for 10 min, and the reaction products were analyzed by 20% polyacrylamide gel electrophoresis containing 8 M urea (Urea-PAGE). The reaction products were then visualized by imaging the gel on the Amersham Typhoon Laser Scanner (Cytiva Life Sciences, Marlborough, MA, USA). At the end of the polymerase-dependent 3′-AZ-dTMP incorporation reaction to the 5′-FAM-45-mer DNA, it resulted in the formation of a 5′-FAM-46-mer DNA carrying an azide group at the 3′-end (shown as lane 2 of FIGS. 2A, 3, and 5, respectively), which can be used for a subsequent labeling reaction. Alternatively, the 5′-FAM-46-mer DNA was further purified using the DNA QIAquick Nucleotide Removal Kit (Qiagen, Germantown, MD) before the subsequent labeling reaction. The preparation of a 3′-azide-labeled target polynucleotide containing a 3′-O-azidomethyl group is shown in Scheme 1 of FIG. 16A.

### Example 2: 3′-Labeling of a Polynucleotide Carrying an Azidomethyl Group at the 3′-End Via an Azide-Alkyne Coupling Reaction

As previously described in Example 1, once the polynucleotide containing an azidomethyl group at the 3′-end is obtained, the 3′-end azide (N3) group can be directly used for labeling with any desired tag or functional molecule, such as a fluorescent dye or quencher, via an azide-alkyne coupling reaction.

For example, when a Cyanine 5 (Cy5) fluorophore-label at the 3′-end of the target polynucleotide is desired, the Cy5-alkyne molecule can be chosen for the azide-alkyne coupling reaction with the polynucleotide carrying an azide (N3) group at the 3′-end. In at least one embodiment, the polynucleotide with a 3′-azide group can directly react with the Cy5-alkyne presence of a tris(3-hydroxypropyltriazolylmethyl)amine (THPTA) ligand, copper ions, and sodium ascorbate to trigger the Cu-catalyzed cycloaddition reaction between the 3′-azide group of the polynucleotide and the alkyne group of the Cy5-alkyne molecule. The azide-alkyne coupling reaction was normally performed at 37° C. for 1 hour. The unreactive Cy5-alkyne molecule was then removed by using the DNA QIAquick Nucleotide Removal Kit (Qiagen, Germantown, MD, USA). The clean-up reaction products were further subjected to the 3′ to 5′ exonuclease (e.g., 200 nM of 3′ to 5′ exonuclease) treatment at 37° C. for 1 hour. The final reaction products were analyzed with 20% Urea-PAGE and visualized by imaging the gel on the Amersham Typhoon Laser Scanner (Cytiva Life Sciences, Marlborough, MA, USA). The results of azide-alkyne cycloaddition and enzyme-digestion reactions generate a homogeneous target polynucleotide containing a Cy5 fluorophore at the 3′-end (referring to lane 3 of FIGS. 2A and 2B). The labeling of a Cy5 fluorophore to the 3′-end of a target polynucleotide is shown in Scheme 2 of FIG. 16B.

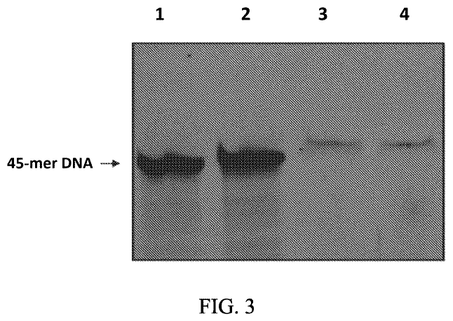

Similarly, when a fluorescent quencher label, such as a Black Hole Quencher 1 (BHQ1), at the 3′-end of a target polynucleotide is desired, the BHQ1-alkyne molecule can be chosen for the azide-alkyne coupling reaction with the target polynucleotide carrying an azide (N3) group at the 3′-end. For example, the polynucleotide with a 3′-azide group was reacted with the BHQ1-alkyne in the presence of a tris(3-hydroxypropyltriazolylmethyl)amine (THPTA) ligand, copper ions, and sodium ascorbate to trigger the Cu-catalyzed cycloaddition reaction between the 3′azide group and the alkyne group of the BHQ1-alkyne molecule. The azide-alkyne coupling reaction was normally performed at 37° C. for 1 hour. The unreactive BHQ1-alkyne molecule was then removed by using the DNA QIAquick Nucleotide Removal Kit (Qiagen, Germantown, MD, USA). The clean-up reaction products were further subjected to the 3′ to 5′ exonuclease (e.g., 200 nM of 3′ to 5′exonuclease) treatment at 37° C. for 1 hour. The final reaction products were analyzed with 20% Urea-PAGE and visualized by imaging the gel on the Amersham Typhoon Laser Scanner (Cytiva Life Sciences, Marlborough, MA, USA). The sequential azide-alkyne cycloaddition and enzyme-digestion reactions generate a homogeneous target polynucleotide containing a BHQ1 label at the 3′-end (referring to lane 4 of FIG. 3). The labeling of a fluorescent quencher to the 3′-end of a target polynucleotide is shown in Scheme 3 of FIG. 16C.

The components and individual experimental groups of the BHQ1-labeling reaction are summarized in Table 1 below. The results of the labeling reaction corresponding to each experimental group are shown in lanes 1 to 4 in FIG. 3, respectively. In Table 1, four experimental groups were designed, and the reaction components are shown, in which the symbol “+” denotes the addition of a designated reagent to the reaction in each experimental group, and the symbol “−” denotes that the designated reagent is not added to the reaction in the experimental group.

TABLE 1

Experimental groups

| Components | (1) | (2) | (3) | (4) |
|---|---|---|---|---|
| 5′-FAM-45-mer DNA polynucleotide | + | + | + | + |
| DNA polymerase | − | + | + | + |
| 3′-AZ-dTTP | − | + | + | + |
| BHQ1-alkyne | − | − | + | + |
| 3′ to 5′ exonuclease | − | − | − | + |

### Example 3: 3′-Labeling of a Polynucleotide Carrying an Azide Group at the 3′-End Via an Azide-DBCO Ligation Reaction

Alternatively, once the polynucleotide having an azidomethyl group at the 3′-end is obtained, the terminal azide group can also be used for labeling with any desired tag or functional molecule, such as a fluorescent dye or quencher, via the azide-dibenzoazacyclooctyne (DBCO) ligation reaction.

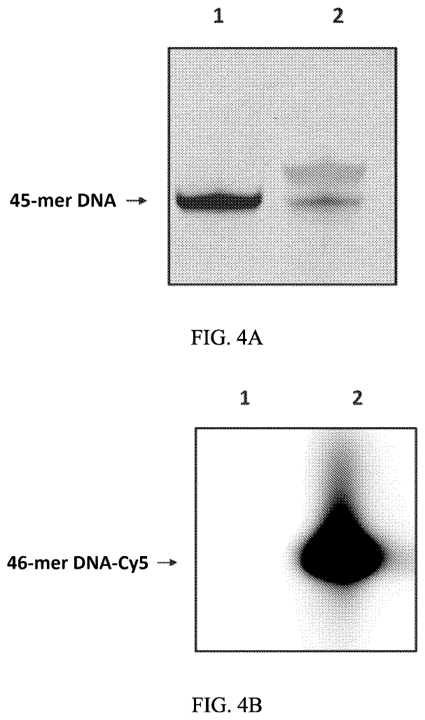

For example, when a Cyanine 5 (Cy5) fluorophore-label at the 3′-end of a target polynucleotide is desired, the Cy5-DBCO molecule can be chosen for the azide-DBCO ligation reaction with the polynucleotide carrying an azide (N3) group at the 3′-end. In at least one embodiment, the polynucleotide with a 3′-azide group can directly react with the Cy5-DBCO in the 1×TE buffer. The azide-DBCO ligation reaction was normally performed at 37° C. for 1 hour. The unreactive Cy5-DBCO molecule was then removed by using the DNA QIAquick Nucleotide Removal Kit (Qiagen, Germantown, MD, USA). The reaction products were analyzed with 20% Urea-PAGE and visualized by imaging the gel on the Amersham Typhoon Laser Scanner (Cytiva Life Sciences, Marlborough, MA, USA). The azide-DBCO ligation reaction generates a target polynucleotide having a Cy5 fluorophore at the 3′-end (referring to lane 2 of FIGS. 4A and 4B). The labeling of a Cy5 fluorophore to the 3′-end of a target polynucleotide is shown in Scheme 4 of FIG. 16D.

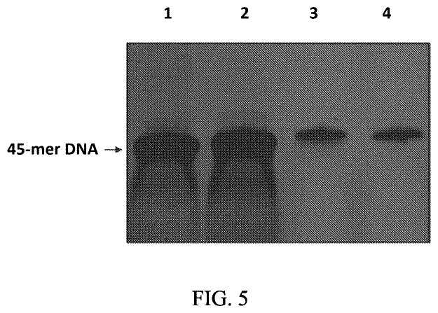

Similarly, when a fluorescent quencher label, such as a Black Hole Quencher 1 (BHQ1) at the 3′-end of a polynucleotide is desired, the BHQ1-DBCO molecule can be used for the azide-DBCO ligation reaction with the target polynucleotide carrying an azide (N3) group at the 3′-end. For example, the target polynucleotide with a 3′-azide group can directly react with the BHQ1-DBCO in the 1×TE buffer. The azide-DBCO ligation reaction was normally performed at 37° C. for 1 hour. The unreactive BHQ1-DBCO molecule was then removed by using the DNA QIAquick Nucleotide Removal Kit (Qiagen, Germantown, MD, USA). The clean-up reaction products were further subjected to the 3′ to 5′ exonuclease (e.g., 200 nM of 3′ to 5′ exonuclease) treatment at 37° C. for 1 hour. The final reaction products were analyzed with 20% Urea-PAGE and visualized by imaging the gel on the Amersham Typhoon Laser Marlborough, MA, USA). The sequential azide-DBCO ligation and enzyme-digestion reactions generate a homogeneous target polynucleotide having a BHQ1 label at the 3′-end (referring to lane 4 of FIG. 5). The labeling of a fluorescent quencher to the 3′-end of a target polynucleotide is shown Scheme 5 of FIG. 16E.

The components and individual experimental groups of BHQ1-labeling reaction are summarized in Table 2. The results of the labeling reaction corresponding to each experimental group are shown in lanes 1 to 4 in FIG. 5, respectively. In Table 2, four experimental groups were designed, and the reaction components are shown, in which the symbol “+” denotes the addition of a designated reagent to the reaction in each experimental group, and the symbol “−” denotes that the designated reagent is not added to the reaction in the experimental group.

TABLE 2

Experimental groups

| Components | (1) | (2) | (3) | (4) |
|---|---|---|---|---|
| 5′-FAM-45-mer DNA polynucleotide | + | + | + | + |
| (5′-FAM-labeled 45-mer DNA) | | | | |
| DNA polymerase | − | + | + | + |
| 3′-AZ-dTTP | − | + | + | + |
| BHQ1-DBCO | − | − | + | + |
| 3′ to 5′ exonuclease | − | − | − | + |

### Example 4: 3′-Labeling of a Polynucleotide Having a Partial Double-Stranded Region

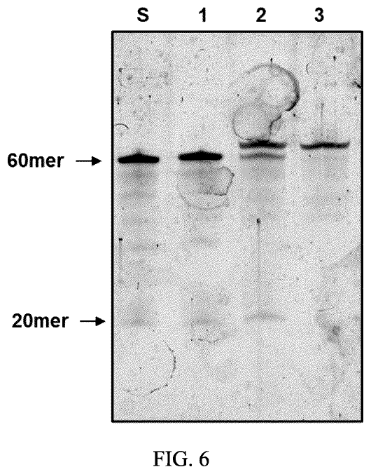

A polynucleotide having a partial double-stranded region can also be used as the target polynucleotide for 3′-modification or labeling by adding a 3′-AZ-dNTP to the 3′-end of the target polynucleotide. For example, a partial double-stranded polynucleotide consisting of a 60-mer forward strand and a 20-mer reverse strand was used as the target polynucleotide. To introduce an azidomethyl group to the 3′-end of the partial double-stranded DNA, the 3′-O-azidomethyl-deoxynucleoside triphosphate (3′-AZ-dNTP) was used as a template-independent DNA synthesis substrate for the selective B-family DNA polymerase or its variant. The nucleotide incorporation reaction was performed in the reaction mixture (10 μL) containing 100 nM of the partial double-stranded target polynucleotide, 0.25 mM of manganese chloride (MnCl₂), and 200 nM of the polymerase. The reaction was initiated by the addition of 25 μM of 3′-O-azidomethyl-dTTP (3′-AZ-dTTP) and then incubated at 60° C. for 10 minutes. The reaction was then terminated by adding a 10 μL of 2× quench solution (95% de-ionized formamide and 25 mM EDTA). The reaction mixture was further denatured at 95° C. for 10 min, which was then subjected to 20% polyacrylamide gel electrophoresis containing 8 M urea (Urea-PAGE). The reaction products were visualized by imaging the gel on the Amersham Typhoon Laser Scanner (Cytiva Life Sciences, Marlborough, MA, USA). After the polymerase-dependent 3′-AZ-dTMP incorporation reaction to the partial double-stranded polynucleotide, formation of a 61-mer forward strand carrying an azide group at the 3′-end was obtained (shown as lane 1 of FIG. 6, wherein lane S shows the electrophoretic location of the partial double-stranded polynucleotide before the 3′-end nucleotide incorporation reaction), which can be used for a subsequent labeling reaction.

In this example, the partial double-stranded polynucleotide containing an azidomethyl group at the 3′-end as obtained above was subjected to a subsequent labeling reaction via an azide-dibenzoazacyclooctyne (DBCO) ligation, as mentioned above.

The result of the labeling reaction of the partial double-stranded polynucleotide is shown in FIG. 6, wherein lane S shows the electrophoretic location of the partial double-stranded polynucleotide before a labeling reaction; lane 1 shows the result of formation of a 61-mer forward strand carrying an azide group at the 3′-end; lane 2 shows the result after the labeling reaction; and lane 3 shows the result after a clean-up reaction, in which the labeled products were further subjected to the 3′ to 5′ exonuclease (e.g., 200 nM of 3′ to 5′exonuclease) treatment at 37° C. for 1 hour.

This example demonstrates the applicability of the 3′-labeling method of the present disclosure to a polynucleotide having a partial double-stranded region, or a DNA consisting of strands of different lengths.

### Example 5: 3′-Labeling of a Polynucleotide with Different 3′-O-azidomethyl Deoxynucleotides

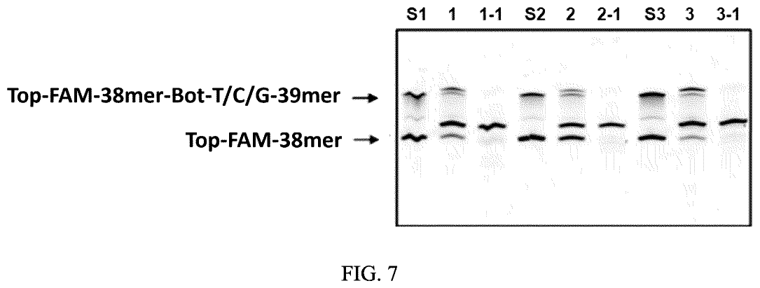

A 38-mer DNA polynucleotide containing a fluorescein label at the 5′-end (5′-FAM-38-mer DNA) was used as the target polynucleotide for 3′-modification or labeling. To introduce an azidomethyl group to the 3′-end of the 5′-FAM-38-mer DNA, different fluorescent-labeled 3′-O-azidomethyl-deoxynucleoside triphosphates including Cy5-labeled 3′-AZ-dATP, Cy5-labeled 3′-AZ-dGTP and IF700-labeled 3′-AZ-dCTP were used as a template-independent DNA synthesis substrate for the selective B-family DNA polymerase used. The exemplary polymerase used herein is a Vent polymerase, which can efficiently incorporate the various 3′-AZ-dNTP to the 3′-end of the target polynucleotide. The nucleotide incorporation reactions were performed in the reaction mixtures (10 μL) containing 100 nM of 5′-FAM-38-mer DNA target polynucleotide, 0.25 mM of manganese chloride (MnCl₂), and 200 nM of polymerase. The reactions were initiated by the addition of 25 μM of Cy5-labeled 3′-AZ-dATP, Cy5-labeled 3′-AZ-dGTP or IF700-labeled 3′-AZ-dCTP, respectively, and then incubated at 60° C. for 20 minutes. The reactions were then terminated by adding a 10 μL of 2× quench solution (95% de-ionized formamide and 25 mM EDTA) to each reaction mixture, which were further denatured at 95° C. for 10 min. The reaction products were then analyzed by 20% polyacrylamide gel electrophoresis containing 8 M urea (Urea-PAGE) and visualized by imaging the gel on the Amersham Typhoon Laser Scanner (Cytiva Life Sciences, Marlborough, MA, USA). After incorporating the Cy5-labeled 3′-AZ-dATP, Cy5-labeled 3′-AZ-dGTP and IF700-labeled 3′-AZ-dCTP to the 5′-FAM-38-mer polynucleotides, 5′-FAM-39-mer polynucleotides were formed, carrying an azide group at the 3′-end (shown as lanes 1, 2 and 3 of FIG. 7, respectively).

In this example, the 5′-FAM-39-mer polynucleotides containing different azidomethyl groups at the 3′-end as obtained above were subjected to a subsequent labeling reaction via a direct incorporation of fluorescent-labeled 3′-O-azidomethyl-deoxynucleoside triphosphates.

The result of the labeling reaction of the partial double-stranded polynucleotide is shown in FIG. 7, wherein lanes S1, S2 and S3 show the electrophoretic locations of the 5′-FAM-38-mer and 5′-FAM-39-mer polynucleotides before a labeling reaction, respectively; lanes 1, 2 and 3 show the electrophoretic locations of the fluorescent-labeled 5′-FAM-38-mer and 5′-FAM-39-mer polynucleotides after the labeling reaction, respectively; and lanes 1-1, 2-1 and 3-1 show the result after a clean-up reaction, in which the labeled products were further subjected to the 3′ to 5′ exonuclease (e.g., 200 nM of 3′ to 5′exonuclease) treatment at 37° C. for 1 hour.

This example demonstrates the applicable uses of different 3′-O-azidomethyl-deoxynucleoside triphosphates to introduce an azidomethyl group to the 3′-end of a target polynucleotide using the 3′-labeling method of the present disclosure.

### Example 6: 5′-End Labeling of Nucleic Acids Using an Aldehyde-Reactive Compound

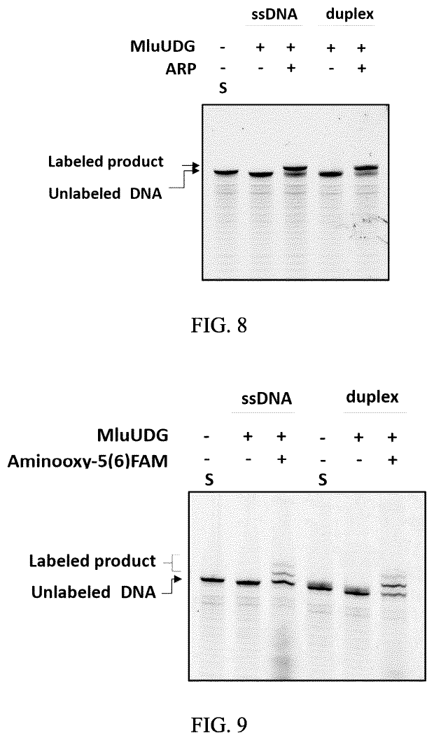

A 45-mer single-stranded DNA (ssDNA; 5′-/deoxyU/CTCGGCCTGGCACAGGTCCGTCTCAGTGCTGCGGCGACCACCG A-3′ (SEQ ID NO: 1)) containing a uracil residue at the 5′-end and a fluorescein (FAM) dye at the 3′-end was synthesized. To carry out the uracil excision and subsequent abasic site labeling, 100 nM of 45-mer uracil-containing ssDNA was mixed with 100 ng of uracil-DNA glycosylase derived from Micrococcus luteus (MluUDG), 5 of mM an aldehyde-reactive probe (N-(aminooxyacetyl)-N′-biotinylhydrazine). The reaction was initiated by the addition of MluUDG and the aldehyde-reactive probe at 37° C. for 15 minutes. The reaction was terminated by adding an equal volume of 2× quench solution (30 mM EDTA and 95% (v/v) de-ionized formamide) and then denatured at 95° C. for 10 min. The reaction products were analyzed by a denaturing 20% polyacrylamide gel electrophoresis containing 8 M urea (Urea-PAGE). The result was visualized by scanning the gel at Amersham Typhoon Imager (Cytiva Life Sciences, Marlborough, MA, USA) and shown in FIG. 8. As shown in the FIG. 8, the addition of MluUDG and the aldehyde-reactive probe produced an additional band with a higher molecular weight in the gel image, indicating the presence of a labeled ssDNA product.

Similarly, in another example, a partial duplex DNA molecule was labeled with an aldehyde-reactive probe and analyzed. The duplex DNA was prepared by annealing the 45-mer uracil-containing ssDNA (SEQ ID NO: 1) to a 15-mer complementary strand (5′-TGTGCCAGGCCGAGA-3′ (SEQ ID NO: 2)) at a molar ratio of 1:1.5 in the 1× Tris-EDTA (TE) buffer consisting of 10 mM Tris-HCl (pH 8.0), 1 mM EDTA, and 100 mM NaCl. The DNA annealing reaction was performed in a thermal cycler by heating up the DNA mixture to 98° C. for 3 minutes, followed by gradually cooling down (e.g., 30 seconds for every 5° C.) to 4° C. The resulting duplex DNA was subjected to the uracil excision by MluUDG and subsequent abasic site labeling as described in the above steps and conditions for labeling ssDNA. The reaction was terminated by adding an equal volume of 2× quench solution and then denatured at 95° C. for 10 min. The reaction products were analyzed by a 20% Urea-PAGE. The result was visualized by scanning the gel at Amersham Typhoon Imager and shown in FIG. 8. In the FIG. 8, the addition of MluUDG and the aldehyde-reactive probe to the duplex DNA generated an additional band with a higher molecular weight in the gel, indicating the presence of a labeled DNA product.

Therefore, both the single-stranded and duplex DNAs were able to be labeled with an aldehyde-reactive probe by the provided method.

### Example 7: 5′-End Labeling of Nucleic Acids Using an aminooxy-5(6)-FAM

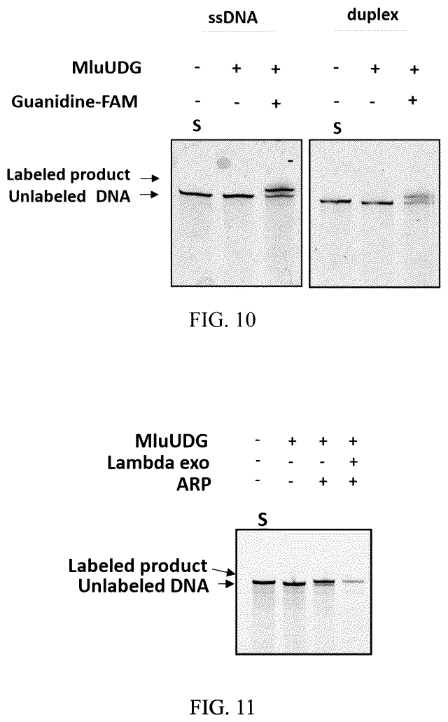

A 45-mer single-stranded DNA (ssDNA; SEQ ID NO: 1) containing a uracil residue at the 5′-end and a Cyanine 5 (Cy5) dye at the 3′-end was synthesized. To carry out the uracil excision and subsequent abasic site labeling, 100 nM of the ssDNA was mixed with 100 ng of uracil-DNA glycosylase derived from Micrococcus luteus (MluUDG) and 2 mM of aminooxy-5(6)-FAM. The reaction was initiated by the addition of MluUDG and aminooxy-5(6)-FAM and incubated at 37° C. for 60 minutes. The reaction was terminated by adding an equal volume of 2× quench solution (30 mM EDTA and 95% (v/v) de-ionized formamide) and then denatured at 95° C. for 10 min. The reaction products were analyzed by a 20% Urea-PAGE. The result was visualized by scanning the gel at Amersham Typhoon Imager. As shown in FIG. 9, the addition of MluUDG and the aminooxy-5(6)-FAM produced additional bands with higher molecular weights in the gel, indicating the presence of labeled FAM-ssDNA products.

Likewise, in another example, a partial duplex DNA molecule was labeled with an aminooxy-5(6)-FAM and analyzed. The duplex DNA was prepared by annealing the 45-mer uracil-containing ssDNA (SEQ ID NO: 1) to a 15-mer complementary strand (5′-TGTGCCAGGCCGAGA-3′ (SEQ ID NO: 2)) at a molar ratio of 1:1.5 in the 1×TE buffer consisting of 10 mM Tris-HCl (pH 8.0), 1 mM EDTA, and 100 mM NaCl. The DNA annealing reaction was performed in a thermal cycler by heating up the DNA mixture to 98° C. for 3 minutes and gradually cooling down (e.g., 30 seconds for every 5° C.) to 4° C. The resulting duplex DNA was subjected to the uracil excision by MluUDG and subsequent abasic site labeling as described in the above steps and conditions for labeling ssDNA. The reaction was terminated, and the products were analyzed by a 20% Urea-PAGE. The result was visualized by scanning the gel at Amersham Typhoon Imager. As also shown in FIG. 9, the addition of MluUDG and the aminooxy-5(6)-FAM to the duplex DNA generated additional bands with higher molecular weights in the gel, indicating the presence of labeled FAM-DNA products.

Hence, both single-stranded and duplex DNAs were shown to be labeled with the aminooxy-5(6)-FAM by the provided method.

### Example 8: 5′-End Labeling of DNA with the Naphthalene- and Guanidine-Containing Aminooxy-FAM

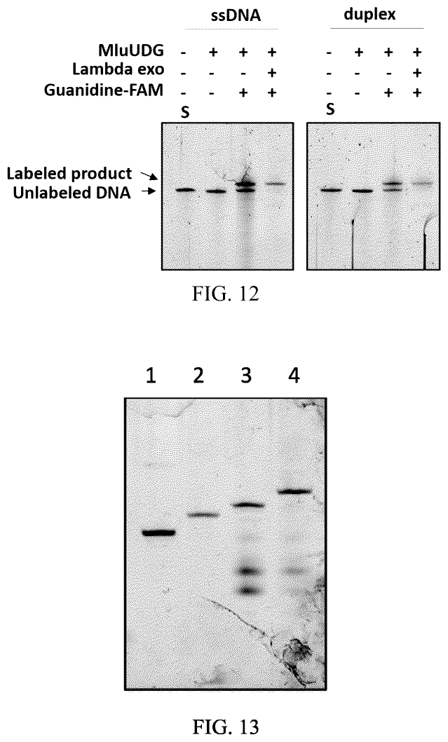

A 47-mer single-stranded DNA (ssDNA; 5′-/deoxyU/CTCGGCCTGGCACAGGTCCGTCTCAGTGCTGCGGCGACCACCG AGG-3 (SEQ ID NO: 3)) containing a uracil residue at the 5′-end was synthesized. To carry out the uracil excision and subsequent abasic site labeling, 100 nM of the ssDNA was mixed with 100 ng of uracil-DNA glycosylase derived from Micrococcus luteus (MluUDG) and 2 mM of naphthalene- and guanidine-containing aminooxy-FAM. The reaction was initiated by the addition of MluUDG and a naphthalene- and guanidine-containing aminooxy-FAM and incubated at 37° C. for 60 minutes. The reaction was terminated by adding an equal volume of 2× quench solution (30 mM EDTA and 95% (v/v) de-ionized formamide) and then denatured at 95° C. for 10 min. The reaction products were analyzed by a 20% Urea-PAGE. The result was visualized by scanning the gel at Amersham Typhoon Imager. As shown in FIG. 10, the addition of MluUDG and the naphthalene- and guanidine-containing aminooxy-FAM generated an additional band with a higher molecular weight in the gel, indicating the presence of a labeled FAM-ssDNA product.

Similarly, in another example, a partial duplex DNA molecule was also labeled with the naphthalene- and guanidine-containing aminooxy-FAM and analyzed. The duplex DNA was prepared by annealing the 47-mer uracil-containing SSDNA (SEQ ID NO: 3) to a 15-mer complementary strand (SEQ ID NO: 2) at a molar ratio of 1:1.5 in the 1×TE buffer consisting of 10 mM Tris-HCl (pH 8.0), 1 mM EDTA, and 100 mM NaCl. The DNA annealing reaction was performed in a thermal cycler by heating up the DNA mixture to 98° C. for 3 minutes and gradually cooling down (e.g., 30 seconds for every 5° C.) to 4° C. The resulting duplex DNA was subjected to the uracil excision and subsequent abasic site labeling as described in the above steps and conditions for labeling ssDNA. The reaction was terminated, and the products were analyzed by a 20% Urea-PAGE. The result was visualized by scanning the gel at Amersham Typhoon Imager. As also shown in the FIG. 10, the addition of MluUDG and the naphthalene- and guanidine-containing aminooxy-FAM to the duplex DNA generated an additional band with a higher molecular weight in the gel, indicating the presence of a FAM-labeled duplex DNA product.

Therefore, both single-stranded and duplex DNAs were able to be labeled with the naphthalene- and guanidine-containing aminooxy-FAM by the provided method.

### Example 9: 5′-End Labeling of 5′-phosphorylated DNA with an Aldehyde-Reactive Compound and the Enrichment of Labeled DNA Using a Phage Lambda Exonuclease

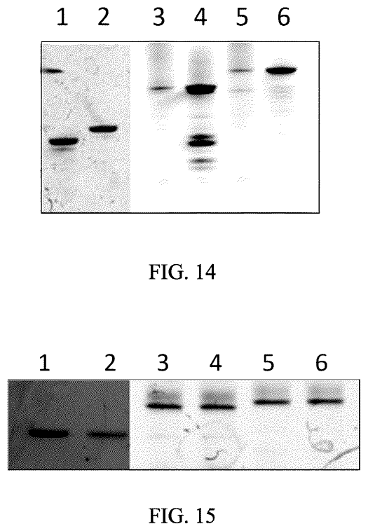

A 45-mer single-stranded DNA (ssDNA; SEQ ID NO: 1) containing a uracil residue at the 5′-end and a fluorescein (FAM) at the 3′-end was synthesized. The DNA was first 5′-end phosphorylated by T4 polynucleotide kinase in the presence of adenosine triphosphate (ATP) at 37° C. for 10 minutes. To carry out the uracil excision and subsequent abasic site labeling, 100 nM of 5′-phosphorylated ssDNA was mixed with 100 ng of uracil-DNA glycosylase derived from Micrococcus luteus (MluUDG) and 5 mM of aldehyde-reactive probe (N-(aminooxyacetyl)-N′-biotinylhydrazine). The reaction was initiated by the addition of MluUDG and the aldehyde-reactive probe at 37° C. simultaneously for 15 minutes. To eliminate the unlabeled ssDNA, 2 units of phage lambda exonuclease were added and then incubated at 37° C. for additional 3.5 hours. The reaction was stopped by adding an equal volume of 2× quench solution (30 mM EDTA and 95% (v/v) de-ionized formamide) and then denatured at 95° C. for 10 min. The reaction products were analyzed by a 20% Urea-PAGE. The result was visualized by scanning the gel at Amersham Typhoon Imager. As shown in FIG. 11, the addition of MluUDG and the aldehyde-reactive probe generated an additional band with a higher molecular weight in the gel, indicating the presence of a labeled ssDNA product. When the reaction mixture was further treated with the phage lambda exonuclease to degrade the unlabeled DNA (i.e., a cleanup step), the portion of labeled ssDNA product was further enriched.

Therefore, DNAs can be labeled with an aldehyde-reactive probe by the provided method, and the labeled DNA fraction can be further enriched by the treatment with the phage lambda exonuclease.

### Example 10: 5′-End Labeling of 5′-phosphorylated DNA with a Naphthalene- and Guanidine-Containing Aminooxy-FAM and the Purification and Enrichment of FAM-Labeled DNA Using Phage Lambda Exonuclease

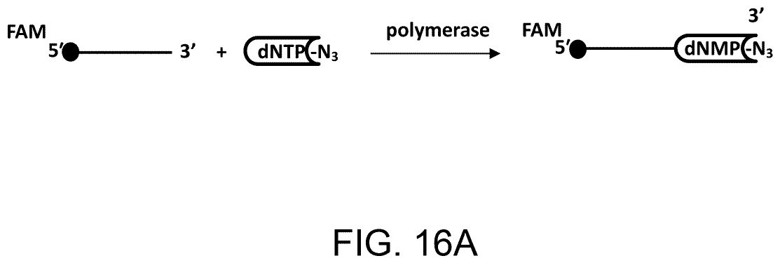

A 47-mer single-stranded DNA (ssDNA; SEQ ID NO: 3) containing a uracil residue at the 5′-end was synthesized. The DNA was first 5′-end phosphorylated by T4 polynucleotide kinase in the presence of adenosine triphosphate (ATP) at 37° C. for 30 minutes. To carry out the uracil excision and subsequent abasic site labeling, 100 nM of the ssDNA was mixed with 115 ng of uracil-DNA glycosylase derived from Micrococcus luteus (MluUDG) and 1 mM of naphthalene- and guanidine-containing aminooxy-FAM. The reaction was initiated by the addition of MluUDG and the naphthalene- and guanidine-containing aminooxy-FAM and incubated at 37° C. for 30 minutes. To eliminate the unlabeled ssDNA, 2 units of phage lambda exonuclease were added and then incubated at 37° C. for additional 30 minutes. The reaction was stopped by adding an equal volume of 2× quench solution (30 mM EDTA and 95% (v/v) de-ionized formamide) and then denatured at 95° C. for 10 min. The reaction products were analyzed by a 20% Urea-PAGE. The result was visualized by scanning the gel at Amersham Typhoon Imager. As shown in FIG. 12, the addition of MluUDG and the naphthalene- and guanidine-containing aminooxy-FAM generated an additional band with a higher molecular weight in the gel, indicating the presence of a FAM-labeled ssDNA product. When the reaction mixture was further treated with the phage lambda exonuclease to degrade the unlabeled DNA, the FAM-labeled ssDNA product was enriched.

Similarly, in another example, a 5′-phosphorylated duplex DNA molecule was also labeled with the naphthalene- and guanidine-containing aminooxy-FAM before subjected to the phage lambda exonuclease treatment to enrich the FAM-labeled duplex DNA. The duplex DNA was prepared by annealing the 47-mer uracil-containing ssDNA (SEQ ID NO: 3) to a 15-mer complementary strand (SEQ ID NO: 2) at a molar ratio of 1:1.5 in the 1×TE buffer consisting of 10 mM Tris-HCl (pH 8.0), 1 mM EDTA, and 100 mM NaCl. The DNA annealing reaction was performed in a thermal cycler by heating up the DNA mixture to 98° C. for 3 minutes and gradually cooling down (e.g., 30 seconds for every 5° C.) to 4° C. The resulting duplex DNA was first 5′-phosphorylated by T4 polynucleotide kinase in the presence of ATP and then subjected to the uracil excision and subsequent abasic site labeling followed with the phage lambda exonuclease treatment as described in the above steps and conditions for ssDNA labeling. The reaction product was analyzed by a 20% Urea-PAGE. The result was visualized by scanning the gel at Amersham Typhoon Imager. As shown in FIG. 12, the addition of MluUDG and the naphthalene- and guanidine-containing aminooxy-FAM to the duplex DNA generated an additional band with a higher molecular weight in the gel, indicating the presence of a FAM-labeled DNA product. When the reaction mixture was further treated with the phage lambda exonuclease to degrade the unlabeled DNA, the FAM-labeled DNA product was enriched.

Therefore, both single-stranded and duplex DNAs can be labeled with the naphthalene- and guanidine-containing aminooxy-FAM by the provided method, and the labeled DNA fraction can be further enriched by the treatment with the phage lambda exonuclease.

### Example 11: Dual Labeling of Double-Stranded DNA with Guanidine-FAM at 5′-End and Direct Incorporation at 3′-End

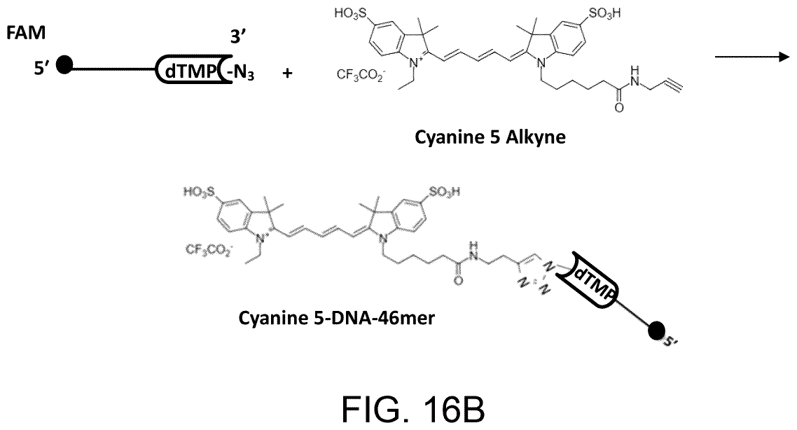

A 21-mer uracil-containing ssDNA was obtained, and its electrophoretic location is shown as lane 1 in FIG. 13. A duplex DNA was prepared from this ssDNA by annealing the 21-mer uracil-containing ssDNA with a 22-mer complementary strand at a molar ratio of 1:1 in the 1×TE buffer consisting of 10 mM Tris-HCl (pH 8.0), 1 mM EDTA, and 100 mM NaCl. The DNA annealing reaction was performed in a thermal cycler by heating up the DNA mixture to 98° C. for 3 minutes and gradually cooling down (e.g., 30 seconds for every 5° C.) to 4° C.

The resulting duplex DNA was subjected to 5′-labeling by reacting 100 nM of the duplex DNA obtained above with 100 ng of MluUDG, 1 mM of guanidine-FAM, and 20 mM of p-phenylenediamine in a reaction mixture of 10 μL at 37° C. for 60 minutes. To eliminate the unlabeled DNA, 1 μM of phage lambda exonuclease was added and then incubated at 37° C. for additional 60 minutes. The reaction was stopped by adding an equal volume of 2× quench solution (25 mM EDTA and 95% (v/v) de-ionized formamide) and then denatured at 95° C. for 10 min. The reaction products were analyzed by a 20% Urea-PAGE. The result was visualized by scanning the gel at Amersham Typhoon Imager. As shown in lane 2 of FIG. 13, the addition of MluUDG and the guanidine-FAM to the duplex DNA shifted the band to a location with higher molecular weights in the gel, indicating the presence of labeled FAM-DNA products.

In a separate reaction, the resulting duplex DNA was subjected to 3′-labeling by direct incorporation of a nucleotide carrying a fluorescent dye. The reaction was performed in a mixture of 10 μL containing 100 nM of the duplex DNA, 200 nM of Vent polymerase, and 20 μM of N3-dTTP-Cy3 at 37° C. for 60 minutes. The result of 3′-labeling is shown as lane 3 in FIG. 13.

A dual labeling reaction was carried out by preparing a mixture of 10 μL containing 100 nM of duplex DNA, 100 ng of MluUDG, 1 mM of guanidine-FAM, 20 mM of p-phenylenediamine, 200 nM of Vent polymerase, and 20 μM of N3-dTTP-Cy3. The reaction was initiated by the addition of MluUDG and the Vent polymerase and incubated at 37° C. for 60 minutes. The result of the duplex DNA dual labeling at both 5′-end and 3′-end is shown as lane 4 in FIG. 13, with a shift of electrophoretic location, indicating the dual-labeled product with a higher molecular weight than both the 5′-end labeled product and the 3′-end labeled product.

### Example 12: Dual Labeling of Double-Stranded DNA with Guanidine-FAM at 5′-End and Azide-DBCO Ligation Reaction at 3′-End

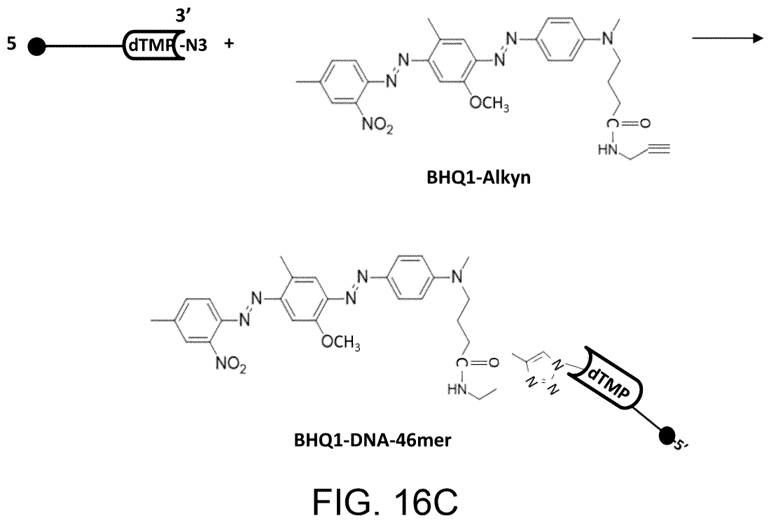

A 26-mer uracil-containing ssDNA was obtained, and its electrophoretic location is shown as lane 1 in FIG. 14. A duplex DNA was prepared from this ssDNA by annealing the 26-mer uracil-containing ssDNA with a 27-mer complementary strand (shown as lane 2 in FIG. 14) at a molar ratio of 1:1 in the 1×TE buffer consisting of 10 mM Tris-HCl (pH 8.0), 1 mM EDTA, and 100 mM NaCl. The DNA annealing reaction was performed in a thermal cycler by heating up the DNA mixture to 98° C. for 3 minutes and gradually cooling down (e.g., 30 seconds for every 5° C.) to 4° C.

The resulting duplex DNA was subjected to 3′-labeling by reacting 100 nM of the duplex DNA obtained above with 200 nM of Vent polymerase, 100 μM of N3-dTTP, and 100 μM of DBCO-Cy5 at 37° C. for 60 minutes. The result of 3′-labeling is shown as lane 3 in FIG. 14. Shift of the electrophoretic location of the 3′-labeled product as compared to the locations in lanes 1 and 2 suggests successful labeling of Cy5. The obtained labeled product was further enriched with a 3′-exonuclease digestion. To remove the unlabeled DNA strand, the reaction was initiated by the addition of 1 μM of hTREX1 in the labeling mixture at 37° C. After 60 minutes, the digestion reaction was stopped by adding 10 μL of 2× quench solution (95% de-ionized formamide and 25 mM EDTA). The result is shown in lane 4 of FIG. 14, which shows a thicker band compared to the band at the same location in lane 3.

A dual labeling reaction was carried out by preparing a mixture of 10 μL containing 100 nM of the duplex DNA in this example, 100 ng of MluUDG, 1 mM of guanidine-FAM, 20 mM of p-phenylenediamine, 200 nM of Vent polymerase, 100 μM of N3-dTTP, and 100 μM of DBCO-Cy5 at 37° C. for 60 minutes. The reaction was initiated by the addition of MluUDG and the Vent polymerase and incubated at 37° C. for 60 minutes. The result of the duplex DNA dual labeling at both 5′-end and 3′-end is shown as lane 5 in FIG. 14, with a shift of the electrophoretic location compared to the bands in lanes 3 and 4, indicating the dual-labeled product with a higher molecular weight.

The dual-labeled product was further subjected to 5′-exonuclease and 3′-exonuclease digestion to remove the unlabeled DNA strand, also called as a clean-up step. The reaction was initiated by the addition of 1 μM of lambda exonuclease and 1 μM of hTREX1 in the labeling mixture above at 37° C. After 60 minutes, the reaction was stopped by adding 10 μL of 2× quench solution (95% de-ionized formamide and 25 mM EDTA). The result is shown in lane 6 of FIG. 14, which shows a thicker band compared to the band at the same location in lane 5, indicating an efficient clean-up of the labeled products.

### Example 13: Dual Labeling of Double-Stranded DNA with Guanidine-FAM at 5′-End and BHQ1 at 3′-End

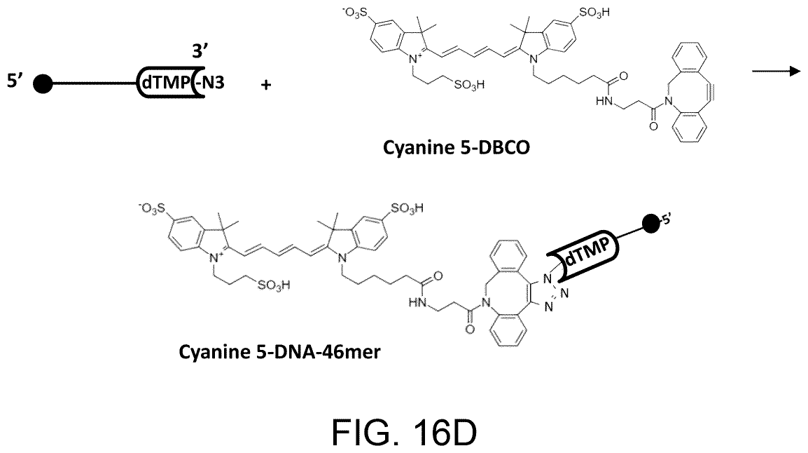

A 25-mer uracil-containing ssDNA was obtained, and its electrophoretic location is shown as lane 1 in FIG. 15. A duplex DNA was prepared from this ssDNA by annealing the 25-mer uracil-containing ssDNA with a 26-mer complementary strand (shown as lane 2 in FIG. 15) at a molar ratio of 1:1 in the 1×TE buffer consisting of 10 mM Tris-HCl (pH 8.0), 1 mM EDTA, and 100 mM NaCl. The DNA annealing reaction was performed in a thermal cycler by heating up the DNA mixture to 98° C. for 3 minutes and gradually cooling down (e.g., 30 seconds for every 5° C.) to 4° C.

The resulting duplex DNA was first subjected to 5′-labeling by reacting 100 nM of the duplex DNA obtained above with 100 ng of MluUDG, 1 mM of guanidine-FAM, and 20 mM of p-phenylenediamine in a reaction mixture of 10 μL at 37° C. for 60 minutes. Then, the 5′-labeling mixture was followed by 3′-labeling by adding to the above mixture with 200 nM of Vent polymerase, 100 μM of N3-dCTP-BHQ1 at 37° C. and incubated for another 60 minutes. Then, to remove the unlabeled DNA strand, the reaction mixture was added with 1 μM of lambda exonuclease and 1 μM of hTREX1 in the obtained labeling mixture at 37° C. After 60 minutes, the reaction was stopped by adding 10 μL of 2× quench solution (95% de-ionized formamide and 25 mM EDTA). The result is shown in lane 3 of FIG. 15, which shows a shift of the band location compared to lanes 1 and 2.

In a separate experiment, simultaneous labeling at 5′-end and 3′-end was carried out by mixing a reaction volume of 10 μL containing 100 nM of the duplex DNA, 100 ng of MluUDG, 1 mM of guanidine-FAM, 20 mM of p-phenylenediamine, 200 nM of Vent polymerase, and 100 μM of N3-dCTP-BHQ1. The reaction was initiated by the addition of MluUDG and the Vent polymerase at 37° C. The reaction was carried out for 60 minutes before being subjected to exonuclease digestion. To remove the unlabeled DNA strand, 1 μM of lambda exonuclease and 1 μM of hTREX1 were added to the labeling mixture at 37° C. and incubated for 60 minutes. The reaction was stopped by adding 10 μL of 2× quench solution (95% de-ionized formamide and 25 mM EDTA). The result of simultaneous dual labeling and exonuclease digestion was shown in lane 4 in FIG. 15. It is shown that the simultaneous reaction does not affect the labeling efficiency of the duplex DNA, with lane 3 and lane 4 showing similar band thickness, which indicates the quantity of the labeled products.

The same procedures above of labeling dsDNA sequentially or simultaneously were carried out again, only substituting the 100 μM of N3-dCTP-BHQ1 in the 3′-end labeling reaction above with 100 μM N3-dCTP and 100 μM of DBCO-BHQ1. After exonuclease digestion, the labeled products were analyzed. As shown in lanes 5 and 6, which show sequential and simultaneous dual labeling at 5′-end and 3′-end, respectively, simultaneous dual labeling reaction involving azide-DBCO ligation at 3′-end has similar labeling efficiency compared to sequential labeling with 5′-end labeling first followed by 3′-end labeling.

The present disclosure has been described with embodiments thereof, and it is understood that various modifications, without departing from the scope of the present disclosure, are in accordance with the embodiments of the present disclosure. Hence, the embodiments described are intended to cover the modifications within the scope of the present disclosure, rather than to limit the present disclosure. The scope of the claims therefore should be accorded the broadest interpretation so as to encompass all such modification.

What is claimed is:

1. A kit for dual labeling of a nucleic acid at 5′-end and 3′-end thereof, comprising:
a 5′-end glycosylase;
an aldehyde-reactive compound;
a nucleotide having a reactive moiety;
a polymerase to incorporate the nucleotide having the reactive moiety to the 3′-end of the nucleic acid; and
a desired molecule having a corresponding functional moiety capable of reacting with the reactive moiety.

2. The kit of claim 1, further comprising at least one of a molecule possessing 5′ to 3′ exonuclease activity and a molecule possessing 3′ to 5′ exonuclease activity to remove an unlabeled target nucleic acid and/or an intermediate nucleic acid.

3. The kit of claim 2, wherein the 5′ to 3′ exonuclease is selected from the group consisting of T5 exonuclease, T7 exonuclease, viral alkaline exonuclease, bacterial alkaline exonuclease, phage lambda exonuclease, exonuclease VIII, RecJ, RecJf, Tth RecJ, Mpn NrnA, human exonuclease 5, human exonuclease 1, SNM1, SNM1A, human SNM1B/Apollo, bovine SNM1B, SXT-Exo, phospholipase D3, phospholipase D4, Sso1391-Csa1, Sto0027-Csa1, Ttx1248-Csa1, Sso1451-Csa1, Sto2633-Csa1, Pfu1793-Cas4, Sto2501, Sso0001, Sto2331-Cas4, Ttx1245-Cas4, Sso1449-Cas4, Sto2635-Cas4, Sso1392-Cas4, SIRV2 gp19, bacterial AddB, and any combination thereof.

4. The kit of claim 1, wherein the desired molecule further comprises a label moiety to form a labeled nucleic acid when the desired molecule is coupled to the 3′-end of the nucleic acid.

5. The kit of claim 1, wherein the polymerase is a template-independent polymerase.

6. The kit of claim 1, wherein the polymerase is a DNA polymerase, an RNA polymerase, or a functionally equivalent enzyme thereof.

7. The kit of claim 6, wherein the DNA polymerase is an A family DNA polymerase, a B family DNA polymerase, or an X family DNA polymerase.

8. The kit of claim 7, wherein the B family DNA polymerase is a Thermococcaceae DNA polymerase.

9. The kit of claim 8, wherein the B family DNA polymerase is a Thermococcus DNA polymerase or a Pyrococcus DNA polymerase.

10. The kit of claim 9, wherein the B family DNA polymerase is selected from the group consisting of a B family DNA polymerase of Thermococcus kodakarensis, a B family DNA polymerase of Pyrococcus furiosus, a B family DNA polymerase of Thermococcus litoralis, a B family DNA polymerase of Thermococcus sp. 9°N, and a B family DNA polymerase of Thermococcus gorgonarius.

11. The kit of claim 6, wherein the DNA polymerase is a modified DNA polymerase.

12. The kit of claim 1, wherein the nucleic acid is linked to an initiator attached to a solid support, and the kit further comprises an endonuclease to enzymatically release the nucleic acid from the initiator.

13. The kit of claim 1, wherein the corresponding functional moiety is capable of reacting with the reactive moiety via a bioorthogonal reaction.

14. The kit of claim 13, wherein the bioorthogonal reaction is click conjugation, oxime/hydrazone formation, Staudinger ligation, tetrazine ligation, or quadricyclane ligation.

15. The kit of claim 14, wherein the click conjugation is selected from the group consisting of copper-catalyzed azide-alkyne cycloaddition, strain-promoted azide-alkyne cycloaddition, isocyanide-based click reaction, and inverse electron demand Diels-Alder reaction.

16. The kit of claim 1, wherein the desired molecule is molecularly recognizable through detection of visible light, fluorescence, photoluminescence, electrochemiluminescence, laser, irradiation, fluorescence resonance energy transfer, fluorogenic conformational change, or fluorescence quenching.

17. The kit of claim 16, wherein the desired molecule is a chemical compound, a fluorescent tag, a dye, a marker, a reporter, a quencher, an amine, an antigen, a ligand, a protein, an antibody, an antibody fragment, a peptide, a peptide analog, or a quantum dot.

18. A method for labeling 5′-end and 3′-end of a nucleic acid, comprising:
providing a target nucleic acid to be labeled;
adding to the target nucleic acid a 5′-end glycosylase;
adding to the target nucleic acid a nucleotide having a reactive moiety;
creating an intermediate nucleic acid having an abasic site at a 5′-end of the target nucleic acid;
incorporating the nucleotide having the reactive moiety to the 3′-end of the intermediate nucleic acid;
providing an aldehyde-reactive compound carrying a detectable label for coupling with the intermediate nucleic acid at the abasic site at the 5′-end;
providing a desired molecule having a corresponding functional moiety capable of reacting with the reactive moiety;
exposing the intermediate nucleic acid to the aldehyde-reactive compound carrying a detectable label; and
exposing the intermediate nucleic acid to the desired molecule having a corresponding functional moiety, thereby forming a labeled nucleic acid with the detectable label coupling with the intermediate nucleic acid at the abasic site to form a labeled nucleic acid with the detectable label attached at the 5′-end and with a linkage formed between the reactive moiety and the corresponding functional moiety.

19. A kit for modifying a 3′-end of a polynucleotide, comprising:
a nucleotide having a reactive moiety;
a polymerase to incorporate the nucleotide having the reactive moiety to the 3′-end of the polynucleotide; and
a desired molecule having a corresponding functional moiety capable of reacting with the reactive moiety.

20. The kit of claim 19, wherein the polymerase is a DNA polymerase, an RNA polymerase, or a functionally equivalent enzyme thereof.

21. The kit of claim 20, wherein the DNA polymerase is an A family DNA polymerase, a B family DNA polymerase, or an X family DNA polymerase.

22. The kit of claim 21, wherein the B family DNA polymerase is a Thermococcaceae DNA polymerase.

23. The kit of claim 22, wherein the B family DNA polymerase is a Thermococcus DNA polymerase or a Pyrococcus DNA polymerase.

24. The kit of claim 19, wherein the corresponding functional moiety is capable of reacting with the reactive moiety via a bioorthogonal reaction.

25. The kit of claim 24, wherein the bioorthogonal reaction is click conjugation, oxime/hydrazone formation, Staudinger ligation, tetrazine ligation, or quadricyclane ligation.

26. A kit for 5′-end labeling of a nucleic acid, comprising a 5′-end glycosylase and an aldehyde-reactive compound.

27. The kit of claim 26, wherein the 5′-end glycosylase is selected from the group consisting of uracil-DNA glycosylase, alkyladenine DNA glycosylase, single-strand-selective monofunctional uracil-DNA glycosylase 1, methyl-binding domain glycosylase 4, thymine DNA glycosylase, MutY homolog DNA glycosylase, alkylpurine glycosylase C, alkylpurine glycosylase D, 8-oxo-guanine glycosylase 1 without an abasic site lyase activity, endonuclease III-like glycosylase 1 without the abasic site lyase activity, endonuclease VIII-like glycosylase 1 without the abasic site lyase activity, endonuclease VIII-like glycosylase 2 without the abasic site lyase activity, endonuclease VIII-like glycosylase 3 without the abasic site lyase activity, enzymatically active fragments thereof, and any combination thereof.

28. The kit of claim 26, wherein the aldehyde-reactive compound is a compound having at least one primary amine, a hydrazide, an acylhydrazide, a compound having an aminooxy (ONH₂) group, a compound having a naphthalene-containing aminooxy group, and/or a compound having a guanidine-containing aminooxy group.

29. The kit of claim 26, further comprising a 5′ to 3′ exonuclease for removing an unlabeled nucleic acid.

30. The kit of claim 29, wherein the 5′ to 3′ exonuclease is selected from the group consisting of T5 exonuclease, T7 exonuclease, viral alkaline exonuclease, bacterial alkaline exonuclease, phage lambda exonuclease, 5′-exonuclease of DNA polymerase I, exonuclease VIII, RecJ, RecJf, Tth RecJ, Mpn NrA, human exonuclease 5, human exonuclease 1, SNM1, SNM1A, human SNM1B/Apollo, bovine SNM1B, SXT-Exo, phospholipase D3, phospholipase D4, Sso1391-Csa1, Sto0027-Csa1, Ttx1248-Csa1, Sso1451-Csa1, Sto2633-Csa1, Pfu1793-Cas4, Sto2501, Sso0001, Sto2331-Cas4, Ttx1245-Cas4, Sso1449-Cas4, Sto2635-Cas4, Sso1392-Cas4, SIRV2 gp19, bacterial AddB, and any combination thereof.


SEQUENCE LISTING (3 sequences)

SEQ ID NO: 1 - DNA, 45 bp, synthetic construct
  source 1..45: mol_type = other DNA
  modified_base 1: mod_base = OTHER; note = uracil
  misc_feature 1: note = RNA
  misc_feature 2..45: note = DNA
tctcggcctggcacaggtccgtctcagtgctgcggcgaccaccga

SEQ ID NO: 2 - DNA, 15 bp, synthetic construct
  source 1..15: mol_type = other DNA
tgtgccaggccgaga

SEQ ID NO: 3 - DNA, 47 bp, synthetic construct
  source 1..47: mol_type = other DNA
  modified_base 1: mod_base = OTHER; note = uracil
  misc_feature 1: note = RNA
  misc_feature 2..47: note = DNA
tctcggcctggcacaggtccgtctcagtgctgcggcgaccaccgagg